(19) United States
(12) Patent Application Publication
(10) Pub. No.: US 2004/0002125 A1
(43) Pub. Date: Jan. 1, 2004

(54) Detection of abnormal cells

(76) Inventors: Peter P. Gombrich, Chicago, IL (US); Matthew Gombrich, Chicago, IL (US); Susan Keesee, Chicago, IL (US); Dan Kusswurm, Chicago, IL (US); Didier Adehl, Chicago (FR); Jennifer Kawaguchi, Chicago, IL (US); Richard A. Domanik, Libertyville, IL (US)

Correspondence Address: MERCHANT & GOULD PC, P.O. BOX 2903, MINNEAPOLIS, MN, 55402-0903, US

(21) Appl. No.: 10/298,269

(22) Filed: Nov. 14, 2002

Related U.S. Application Data
(63) Continuation of application No. 10/114,895, filed on Apr. 1, 2002, now abandoned.
(60) Provisional application No. 60/280,239, filed on Mar. 30, 2001.
(60) Provisional application No. 60/280,216, filed on Mar. 30, 2001.

(51) Int. Cl. G01N 33/574

(52) U.S. Cl. 435/7.23

(57) ABSTRACT

Cellular samples such as cervical cells can be analyzed using a unique combination of markers that identify abnormal cells while subtracting out false positives that would otherwise be generated by, for example, normally proliferating cells and non-cellular debris. Dysplastic cervical cells can thus be identified quickly and accurately. An assay including analytical reagents and methods of their use can be employed in classifying cervical cytology specimens as being normal or abnormal. This assay is configured to be performed using any of several commonly available classes of laboratory instrumentation and upon the entire range of cervical cytology specimen preparations encountered in clinical practice. This assay is particularly intended for use in high volume screening environments, but also has both diagnostic and research applications.

### CROSS-REFERENCE TO RELATED APPLICATIONS

This application is related to and claims priority to Serial No. 60/280,239, filed Mar. 30,2001, entitled “DETECTION AND DIFFERENTIATION OF CANCEROUS CELLS”, and Serial No. 60/280,216, filed Mar. 30, 2001, entitled “MARKER COCKTAIL AND USE IN IDENTIFYING CERVICAL CELL ABNORMALITIES”, which applications are specifically incorporated by reference herein.

### TECHNICAL FIELD

The invention relates generally to analyzing cellular material and relates more specifically to analyzing cervical cells. In particular, the invention relaters to techniques for detecting cervical abnormalities. More particularly, the invention relates to a process whereby cervical cellular samples can be rapidly evaluated for the presence of cellular abnormalities that are indicative of cancerous or precancerous states.

### BACKGROUND

Cervical cancer is the second most common form of cancer among women and can be caused by common papilloma viruses that can be spread through sexual contact. In the United States alone, there are believed to be more than 2,000,000 cases each year of precancerous cervical abnormalities, 65,000 cases of cervical carcinoma in situ, and 15,800 cases of invasive cervical cancer. Outside the U.S., where screening is less common, there are more than 450,000 cases of cervical cancer each year.

Cervical cancer often begins as a precancerous lesion on the cervix, known as a cervical intraepithelial neoplasia. If left untreated, these lesions can deepen over time, and may eventually develop into a dangerous invasive cancer. Fortunately, in cases where abnormalities are detected early, and appropriate intervention occurs, the cure rate for cervical cancer is very high. It is therefore desirable that certain sectors of the female population be regularly screened for cervical cancer.

The American Cancer Society, the National Cancer Institute and the College of Obstetrics and Gynecology recommend annual screening of all sexually active females for cervical abnormalities. The number of such females in the U.S. is believed to approach 100 million. However, the number of tests performed annually in the U.S. is just over 60 million, which leaves nearly half of the potential domestic target population untested. Outside the United States, regular screening for cervical abnormalities is not nearly as prevalent. Thus, many more women are not regularly tested.

The PAP test, developed in the 1940's by Dr. George N. Papanicolaou, is presently the most widely used screening technique for spotting abnormalities in cervical cells. For the traditional PAP test, a sample of cervical cells is collected as part of a woman's routine medical gynecological examination. Cells can be scraped from the cervix using a spatula, brush or other type of collection device.

After a sample is collected, the cells can be transferred from the collection device onto a glass slide by wiping the collection device over the surface of the slide. The cells are then fixed to preserve their morphology; stained with a mixture of chromatic stains called the “PAP stain” to improve visualization of the cells and their structures; and examined under a microscope to detect and classify any abnormal cells that may be present.

This traditional method of slide preparation can frequently result in a non-uniform distribution of cells over the surface of the slide with many cells being present in clumps and clusters rather than being well dispersed. Materials such as mucus and red blood cells may also be transferred from the collection device to the slide via this process. These factors can complicate or even preclude the visual evaluation of the cells on the slide and degrade the performance and reliability of the PAP test.

Cervical cytology specimens can be stained with a mixed chromatic stain (PAP stain) to enhance the visual contrast between the various elements of the cells and can be “screened” under a microscope by a cytotechnologist. During the screening process, the cytotechnologist visually evaluates each cell in the sample, and provisionally classifies the sample as being either normal or abnormal on the basis of cell morphology. During this classification process, the cytotechnologist also considers the medical history of the patient because factors such as patient age, medications and the date of the last menstrual period can significantly influence the cell morphology and can lead to erroneous classification if not taken into account.

The morphological criteria used for visual cell classification have been codified in the US as the “Bethesda” system. Specimens provisionally classified as being abnormal by the cytotechnologist are evaluated microscopically by a pathologist, again on the basis of cell morphology, for the purposes of assigning the final specimen classification and diagnosis. The classification of each specimen and the diagnoses for any specimens that were classified as being abnormal are communicated to the attending physicians who, in turn, provides this information to their patients and, in the case of abnormality, provides the necessary follow-up and treatment.

Automated image analysis systems have also been developed for this purpose. These automated systems use features such as the gross visual morphological characteristics described in the Bethesda system that are mathematically extracted and/or computed from the cell images to classify cells and specimens as normal or abnormal. Again, final classification and diagnosis of abnormal specimens is performed on the basis of the visual evaluation of morphological features by a pathologist viewing the specimen under a microscope.

Visually screened PAP tests have a significant error rate with respect to both false negative and false positive classifications. False negatives represent the failure to detect the presence of an existing cancer or precancerous condition. A false positive result has a significant psychological impact upon the patient and has an economic impact in that it results in unnecessary, but expensive, additional diagnostic testing and sometimes even treatment. The economic impact is particularly critical in public health programs and in developing countries where the funds for cervical cancer screening and treatment are severely limited.

FDA approved automated cervical cancer screening systems have been documented to produce lower false negative and false positive rates than does the manual test. The skill level required of the operator of such a system is also significantly less than that of a cytotechnologist. However, the presently available automated screening systems are very expensive pieces of major laboratory equipment that require a substantial supporting infrastructure for operation and a high volume of samples in order to be economically viable.

It is therefore desirable to provide a means of cervical screening that can produce a determination of whether a sample does or does not contain evidence of cellular abnormalities within the time frame of a typical cervical examination. As such a means can provide results before the patient leaves the examination area, the uncertainty and stress of waiting for a negative diagnosis is eliminated and patients showing positive results can be retained for immediate follow-up and treatment.

Thus, a need remains for a way to quickly and easily screen cellular samples. A need remains for means suitable for rapidly testing a cellular sample for the presence of possible abnormal cells.

### SUMMARY

Cellular samples, such as samples of cervical cells, can be analyzed using a combination of reagents that identify abnormal cells while providing a mechanism for subtracting out material mistakenly identified as abnormal cells, such as normally proliferating cells and non-cellular debris. Dysplastic or otherwise abnormal cervical cells can thus be identified quickly and accurately. The present invention provides means for relatively unskilled personnel to rapidly differentiate between normal and abnormal cervical cytology specimens in a cost effective manner with an accuracy that is comparable to or better than that normally associated with the screening of cytological smears by a cytotechnologist. Furthermore, the present invention can be implemented in a variety of formats that can be adapted to the particular needs of different clinical settings.

A variety of markers can be used to identify abnormal cells. The reagent recognizing a marker can be a single chemical or a mixture of several chemicals. In a particular embodiment, the reagent is a mixture of chemicals. Individual reagents can be delivered to a cell sample simultaneously as a mixture or can be delivered sequentially. The staining reagent or reagents can be selected to bind selectively to particular cells and provide these cells with a detectable signal different in quantity and or quality from the signal provided by other cells. For example, the reagent or reagents can label the particular cells so that they fluoresce at a level or wavelength different than that observed for other cells.

An embodiment of the present invention is found in a method for identifying abnormal cells in a cytological sample. A cytological sample is provided and is contacted with a first reagent that selectively labels proliferating cells. The cytological sample is contacted with a second reagent that selectively labels normal proliferating cells and with a third reagent that selectively labels cellular material. Any portions of the cytological sample that have been labeled by the first reagent and the third reagent but not by the second reagent are considered to be abnormal.

Another embodiment of the present invention is found in a method for detecting abnormal cells in a cytological specimen. The cytological specimen is treated with a first reagent that specifically labels a cellular constituent that differs in concentration between normal and abnormal cells, a second reagent that specifically labels cell types that are not of interest, said cell types being capable of expressing the cellular constituent labeled by the first reagent, and a third reagent that permits discrimination between cellular and non-cellular material.

An image of the cytological specimen after treatment is captured that permits discrimination between the respective areas labeled by the three reagents and the image is evaluated to localize and define objects stained with the first, second and/or third reagents. Cells that have been labeled with the first reagent and the third reagent, but not with the second reagent, are classified as being abnormal.

Another embodiment of the present invention is found in a method for detecting dysplastic cells in a cervical cytological sample. A cervical cytology sample is collected and a cell suspension is formed therefrom. A representative sampling of the cell suspension is deposited on a microscope slide and is subsequently fixed.

The cytological sample is contacted with a cytological detection cocktail that includes a first reagent that identifies proliferating cells, a second reagent that identifies proliferating non-dysplastic cells, and a third reagent that identifies cellular material. Any cells that have been identified by the first reagent and the third reagent but not by the second reagent are considered to be dysplastic.

Another embodiment of the present invention is found in a method for detecting dysplastic cells in a cervical cytological sample. A cervical cytology sample is collected using a cell collection device, and the collected cells are fixed on the cell collection device. The cells are contacted with a cytological detection cocktail that includes a first reagent that labels proliferating cells, a second reagent that labels proliferating non-dysplastic cells, and a third reagent that labels cellular material. Any cells that have been identified by the first reagent and the third reagent but not by the second reagent are considered to be dysplastic.

An embodiment of the present invention is found in a method for detecting abnormal cells in a cytological specimen by treating the cytological specimen with a first reagent that specifically labels a cellular constituent that differs in concentration between normal and abnormal cells, a second reagent that specifically labels cell types that are not of interest, said cell types being capable of expressing the cellular constituent labeled by the first reagent, and a third reagent that permits discrimination between cellular and non-cellular material.

The treated cytological specimen is passed through a flow cytometer in order to capture the fluorescent emissions from each individual object in the specimen in at least three channels, one channel corresponding to the emissions from one of the three reagents with which the specimen was treated. The signals in the three channels are evaluated to estimate the relative levels of staining of the object by the first, second and third reagents, and then cells that have been labeled with the first reagent and the third reagent, but not with the second reagent are classified as being abnormal.

Another embodiment of the present invention is found in a method for selectively identifying dysplastic cervical cells. A cytological sample is contacted with a cytological detection cocktail that includes a first reagent that preferentially stains proliferating cervical cells, a second reagent that preferentially stains normal endocervical cells, and a third reagent that preferentially stains cellular material.

An image of the cytological sample is captured to form a captured cytological image and is processed to identify any dysplastic cells. Any portions of the cytological sample that have not been stained by the third reagent are excluded from consideration as being non-cellular material. Any portions that have been stained by the first reagent are selected for consideration as being proliferating cells. Any portions of the cytological sample that have been stained by the first reagent and the second reagent are excluded from consideration as being normal endocervical cells. Any portions stained or identified by the first and third reagents, but not the second reagent, are identified as containing dysplastic cells.

An embodiment of the present invention can be found in a cytological detection cocktail for selectively identifying dysplastic cells in the presence of non-dysplastic cells. The cytological detection cocktail includes a reagent that selectively and differentially stains proliferating cells, a reagent that selectively and differentially stains proliferating non-dysplastic cells, and a reagent that selectively and differentially stains cellular material. Each of the three reagents can be spectrally differentiated from one another.

Other features and advantages of the present invention will be apparent from the following detailed description and drawings.

### BRIEF DESCRIPTION OF THE FIGURES

FIG. 1 is a photograph illustrating transferrin immuno-staining of a group of abnormal cells, from a case diagnosed as HSIL.

FIG. 2 is a photograph illustrating Papinicolaou stain of the abnormal group of FIG. 1, stained following immuno-staining.

FIG. 3 is a photograph illustrating normal squamous cells that have been stained with anti-epidermal growth factor receptor IgG-PE, taken at 100×.

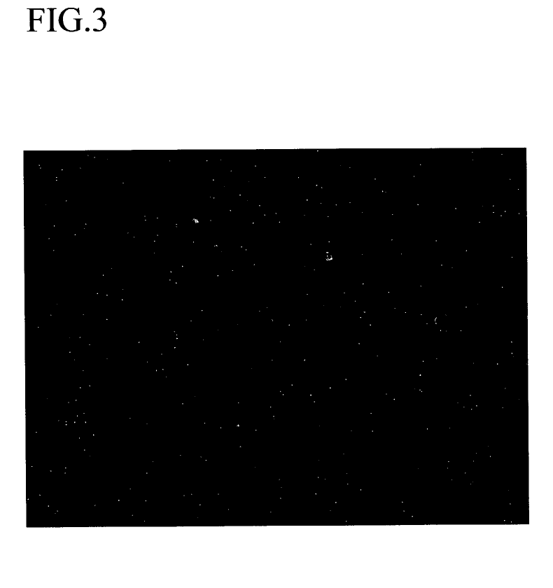

FIG. 4 is a photograph illustrating the cells of FIG. 3, with the addition of anti-cytokeratin 7/8 CAM 5.2-FITC, taken at 100×.

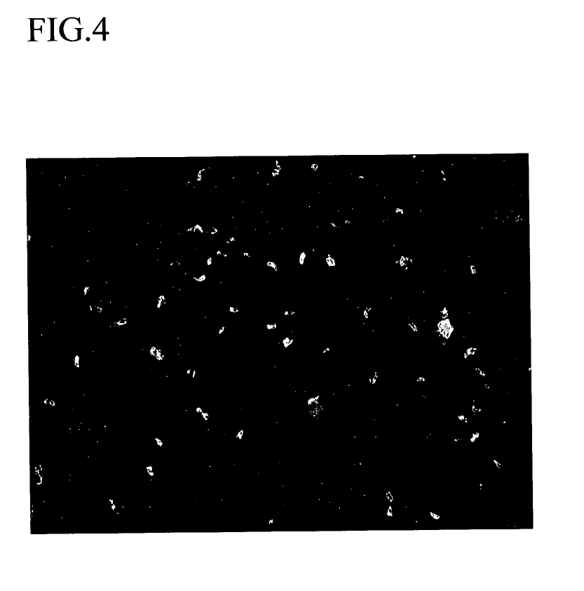

FIG. 5 is a photograph illustrating dysplastic cells (HSIL) that have been stained with anti-EGFR-PE, taken at 100×.

FIG. 6 is a photograph illustrating the dysplastic cells of FIG. 5, taken at 100× after Papinicolaou staining.

FIG. 7 is a photograph illustrating cells from an abnormal Papinicolaou test that have been stained with transferrin receptor, taken at 100×.

FIG. 8 is a photograph of the cells of FIG. 7, with the addition of CAM 5.2-FITC.

FIG. 9 is a photograph of endocervical cells that have been stained with anti-CAM 5.2-FITC, taken at 100×.

FIG. 10 is a photograph of the endocervical cells of FIG. 9, after Papinicolaou staining, also taken at 100×.

FIG. 11 is a photograph of endocervical cells that have been stained with anti-CAM 5.2-FITC, taken at 100×.

FIG. 12 is a photograph of the endocervical cells of FIG. 11, after Papinicolaou staining, also taken at 100×. The cells include metaplastic cells that have not been stained by the anti-CAM 5.2-FITC.

FIG. 13 is a photograph of cervical cell sample that have been stained with DAPI, anti-CAM 5.2-FITC and anti-transferrin receptor-PE, taken at 100×. The arrow indicates a cell of neoplastic origin.

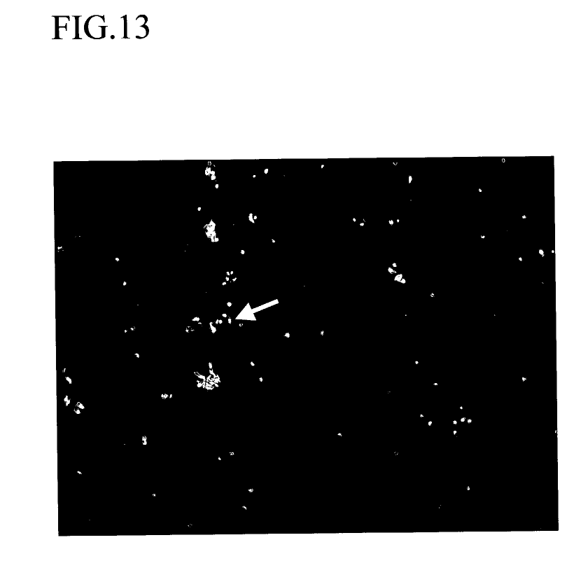

FIG. 14 is a photograph of the cervical cell sample of FIG. 13, after Papinicolaou staining, taken at 100×.

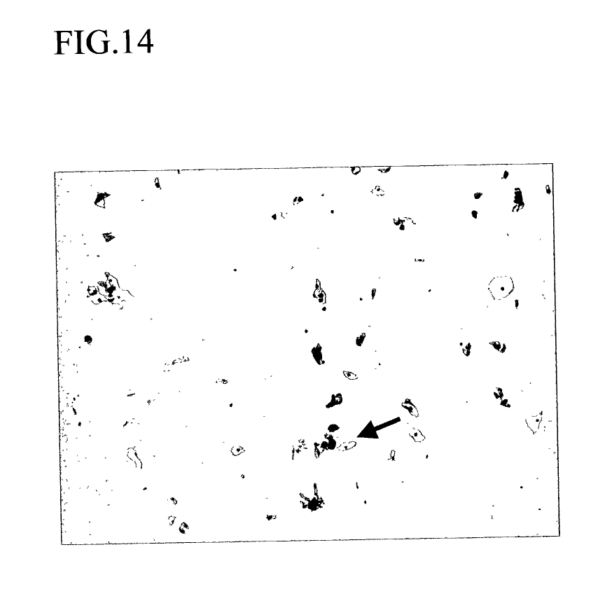

FIG. 15 is a photograph of another cervical cell sample that have been stained with DAPI, anti-CAM 5.2-FITC and anti-transferrin receptor-PE, taken at 100×. The arrow indicates a cell of neoplastic origin.

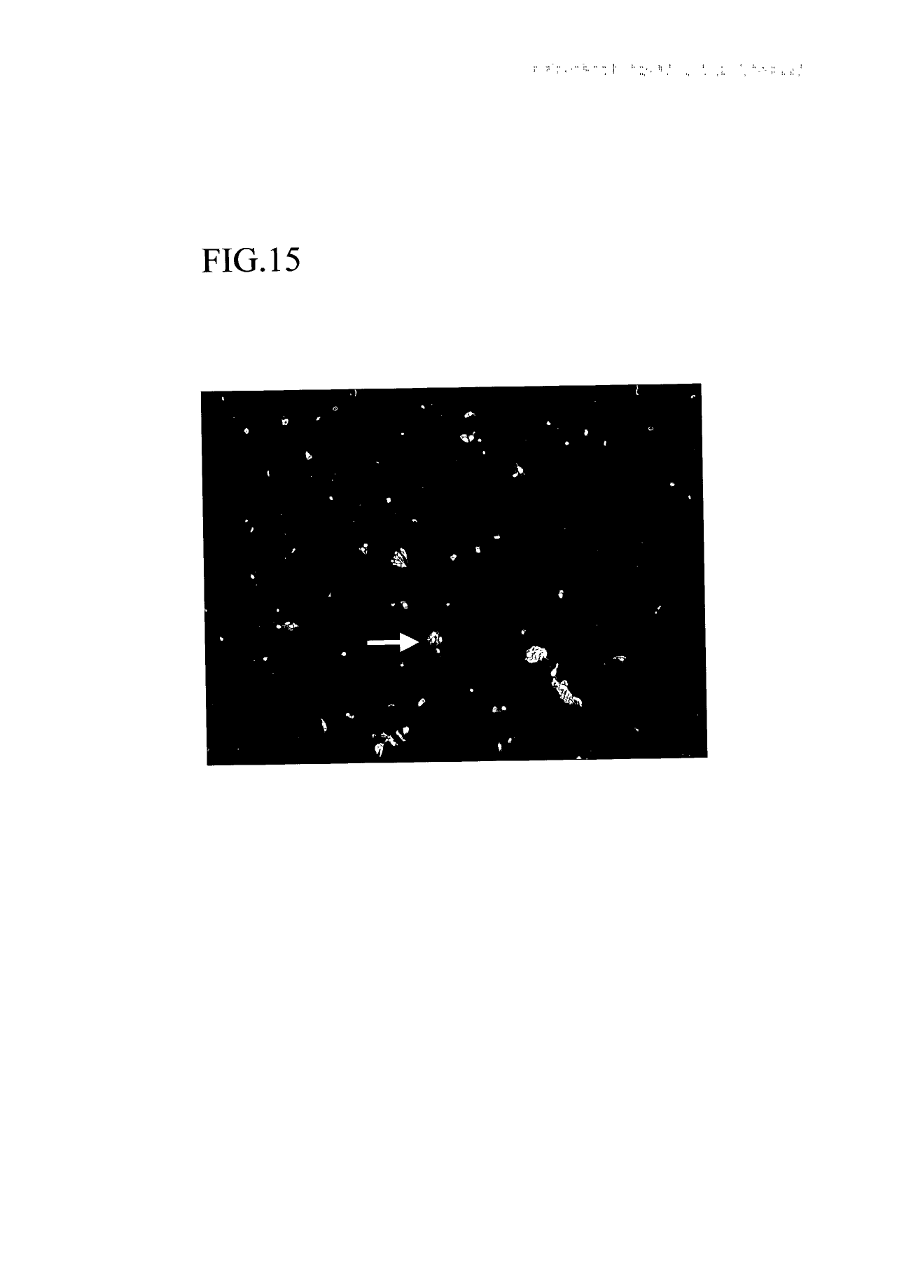

FIG. 16 is a photograph of the cervical cell sample of FIG. 15, after Papinicolaou staining, taken at 100×.

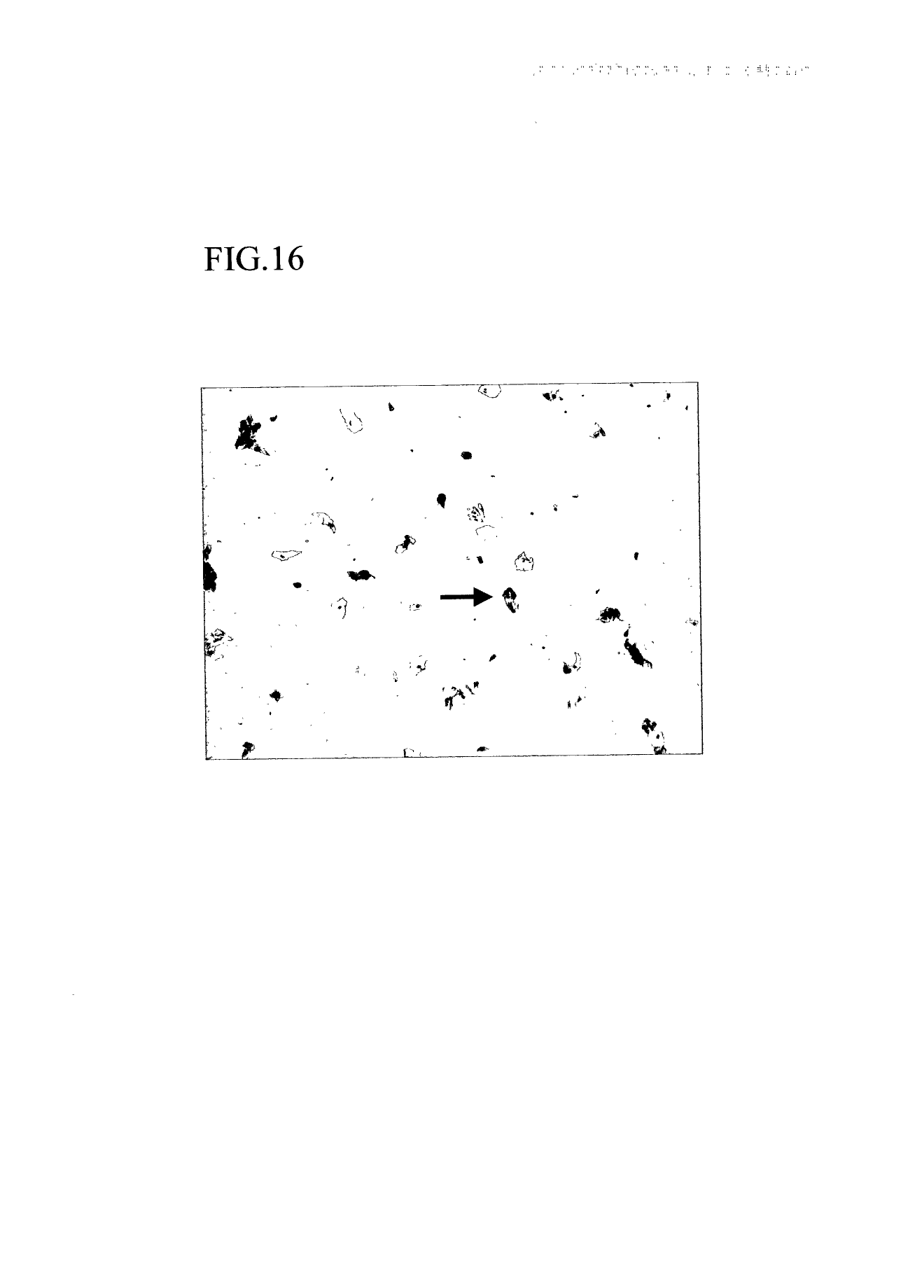

FIG. 17 is a scatter plot of fluorescent emissions for a sample diagnosed as HSIL.

FIG. 18 is a scatter plot of fluorescent emissions for a sample diagnosed as WNL.

FIG. 19 is a scatter plot of fluorescent emissions for another sample diagnosed as HSIL.

FIG. 20 is a scatter plot of fluorescent emissions for another sample diagnosed as WNL.

FIG. 21 is a schematic illustration of a cell transfer device.

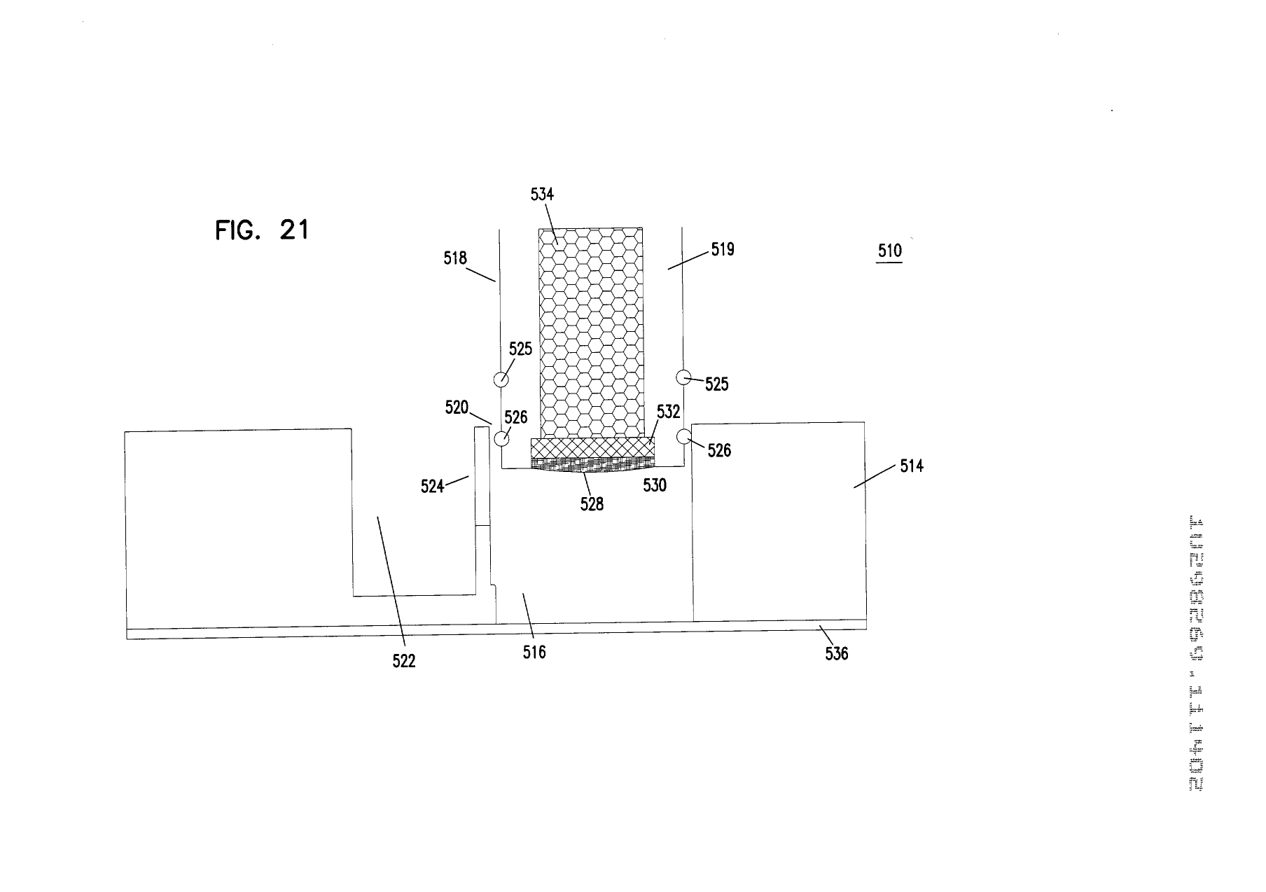

FIG. 22 is a sectional view of a physician's collector, showing the compliant cell sampling member in a folded state prior to use.

FIG. 23 is a sectional view of the physician's collector of FIG. 22, with a handle attached and a container positioned over the sampling member.

FIGS. 24-27 are sectional views of the physician's collector of FIG. 22, illustrating the use of the physician's collector in sampling cervical cells.

FIG. 28 is a sectional view of another embodiment of the sampling member.

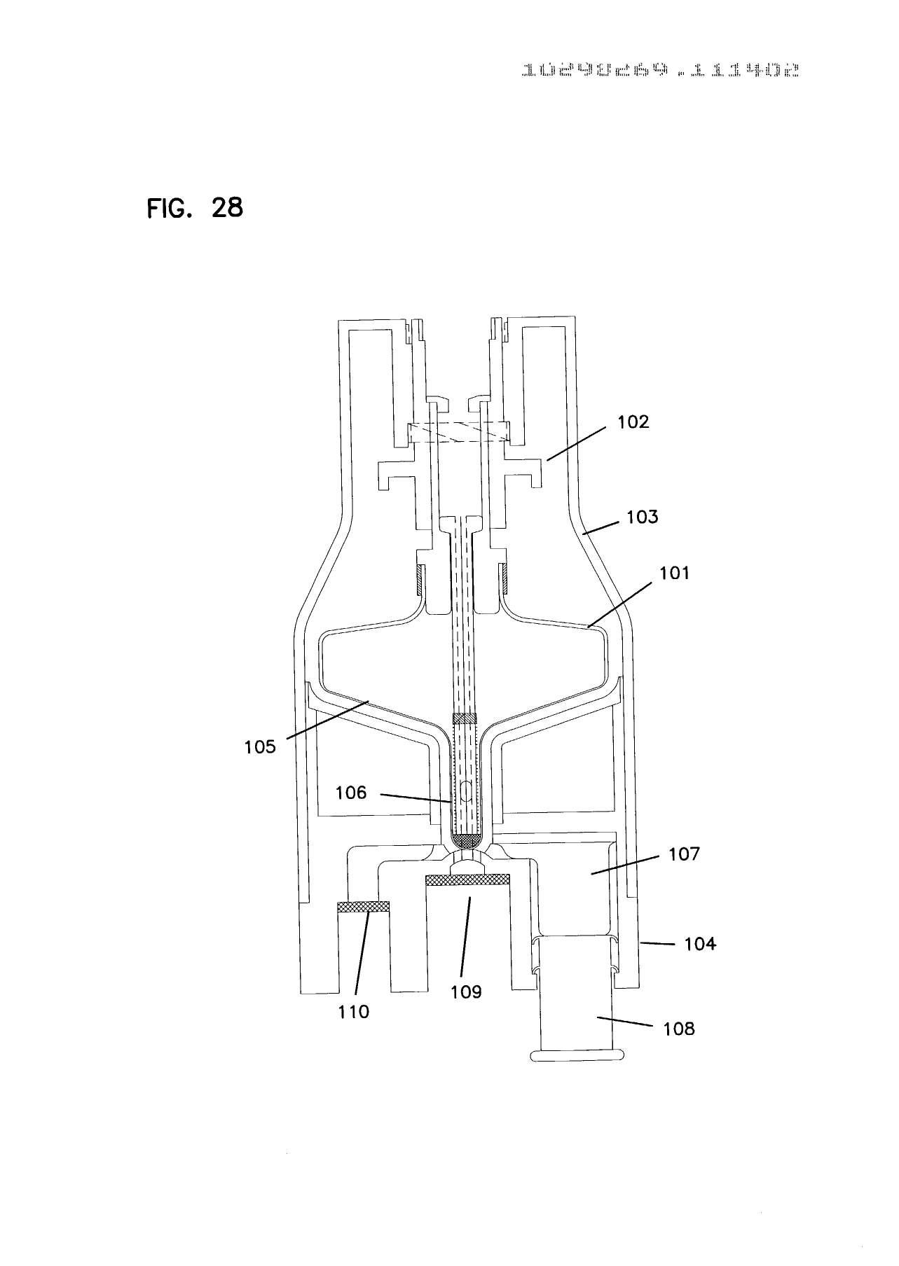

FIG. 29 is a plan view of an analysis instrument.

FIG. 30 is a sectional view of the sample processing station.

### DETAILED DESCRIPTION

Many squamous cell cancers of the cervix originate as squamous interepithelial lesions (SIL) in the transformation (or transition) zone that marks the junction between the endo and ecto-cervical regions. As basal cells in this zone mature, they migrate into the epithelial layer and differentiate into either the glandular cells that line the endocervical canal or into squamous epithelial cells that form the external surface of the ectocervix. Under normal circumstances, these cells serve as replacements for endo and ectocervical epithelial cells that have reached senescence and been exfoliated.

Basal cells can exhibit a high level of metabolic activity that decreases to a maintenance level as the cells mature. The exfoliated cells that comprise a typical cervical cytology sample from a normal cervix can include a mixture of multiple cell types such as mature squamous epithelial cells from the ectocervix; mature endocervical cells from the endocervical canal; a mixture of young squamous epithelial and endocervical cells from the transformation zone and several other minor cell types.

Under circumstances where a high level of cell replacement is occurring, exfoliated metaplastic cells, which are a form that is intermediate between basal cells at one extreme and ecto or endo-cervical cells at the other, may be present in the sample. A similar process can occur at the site of an injury to the cervical epithelium. In either case, these cells represent a continuum of metabolic activities from the highly active basal cells to the nearly inactive mature squames. Furthermore, a mature cell that has suffered an insult or injury will typically increase its metabolic activity during the repair process.

The biochemical events that occur during the transformation of a normal cell into an abnormal cell are, with very few exceptions, identical to those that occur in normal cells of the same type and that the differences between normal and abnormal cells are primarily a matter of location, timing and/or degree of these events. A test capable of differentiating between normal and abnormal cells should, therefore be able to detect the rare unique event that triggered the transformation; discriminate between the locations, timings and/or degrees of events that are affected by the transformation; or detect the results of the transformation. The traditional PAP test, for example, is in this last class as it detects the changes in cell morphology that are associated with various stages of the transformation process.

Another factor that must be considered in the development of such a test is that a significant portion of the cells that exhibit morphological abnormalities detectable using the PAP test will regress to normal without any intervention. For example, considerable evidence currently exists that approximately 80% of the low grade squamous interepithelial lesions (LSIL) diagnosed using the PAP test will revert to normal or, at least, will not progress to overt cancer. On the other hand, the majority of the lesions diagnosed as being high grade squamous interepithelial lesions (HSIL) will ultimately progress to invasive cancer. This consideration permits dividing tests cervical cellular abnormalities into three general, but not mutually exclusive categories: tests that detect the presence of abnormalities; tests that identify the degree of abnormality; and tests that have prognostic value in that they indicate the probability that a given abnormality will progress to overt cancer.

The first of these is of use primarily in the screening for cervical cancer and abnormalities while the latter two are of greatest use in patient management. The present invention is primarily intended for the rapid and cost effective detection of cellular abnormalities, but it can provide some discrimination between degrees of abnormality and, when used in conjunction with certain other tests such as the test for HPV (human papilloma virus) described in U.S. Pat. No. 6,329,152 (which is incorporated by reference herein), can have prognostic utility.

The present invention treats the cervical cytology specimen with at least one and preferably a combination of biochemical and/or chemical reagents, each such reagent which selectively interacts with and binds to or labels a specific cellular constituent, the cellular concentration of which changes as a cell progresses from being normal to being abnormal. Quantitation of these bound materials on either a cell-by-cell or bulk sample basis provides a means of differentiating between normal and abnormal specimens.

The goal of developing a simple, reliable, inexpensive screening test for cervical cellular abnormalities imposes a number of stringent requirements on the design of the test. For example, the cellular constituents targeted by the test should be present, at least in abnormal cells, at concentrations that are sufficiently high as to be readily detected and quantitated using low cost detection devices. Similarly, these targets should be present and readily accessible on the external surface of the cell membrane. This both simplifies and speeds the test by eliminating the need to permeabilize the cell membrane and by eliminating the kinetic penalty associated with requiring the detection reagent to diffuse through the permeabilized membrane.

It is furthermore desirable that the screening test not disrupt the cell morphology or the ability of the cells to be stained with visualization reagents such as a conventional Pap stain. One benefit is that it becomes possible to confirm the results of the screening test and to generate a morphology-based diagnosis on exactly the same sample as was used for screening. This eliminates the uncertainties associated with performing the screening and confirmatory/diagnostic test on different samples or on different portions of the same sample.

The concentrations and/or distributions of many cellular constituents are known to change as a cell undergoes the progression from normal to cancerous. Of these constituents, many are localized in the interior of the cell and therefore can be of less utility in a simple, rapid screening test than are ones that present on the exterior of the cell. Of the constituents that present on the surface of the cell, some are present in low to very low concentrations or exhibit only small changes in concentration as the cell transitions from normal to abnormal. These present detection challenges that are not desirable in the development of a simple, rapid, low cost test. Still others are present at elevated concentrations in only certain stages of the progression to abnormality. While these cellular constituents can be of use in a diagnostic test, they are not necessarily beneficial in a screening test. The relationship between the constituent and the transformation process is also an important consideration, a constituent that is intimately involved in or closely linked to the transformation being preferred to one that is only incidentally involved.

Definitions

As used herein, the term “dysplastic cell” refers to a cell that is precancerous, is abnormal in development or that has an alteration in size, shape, and/or organization.

As used herein, the term “proliferating cell” refers to cells dysplastic, healthy, abnormal or otherwise that are proliferating. A proliferating cell can be a cell that is proliferating as a consequence of an abnormality within the cell. A proliferating cell can be a cell that is proliferating as a consequence of normal, healthy activity within that cell. Such a cell is considered to be a normal proliferating cell.

As used herein, the term “non-dysplastic cell” refers to a cell that is not dysplastic and does not exhibit any features, tendencies or behaviors of a dysplastic cell.

As used herein, the term “debris” refers to noncellular portions of a cervical cell sample, including parts of cells independent of an intact cell (e.g., membrane fragments), fibrous but noncellular tissue, dust, contaminants, and portions of any collector used to collect the cervical cell sample.

As used herein, the term “marker” refers to a biochemical marker or a specific molecule of character that can be detected in a biochemical test and that, typically, is characteristic of a tissue or cell type.

As used herein, the term “identifying” refers to detecting, sensing, labeling, or otherwise making observable and observing a marker or biomolecule.

As used herein, “transferrin receptor”, also known as CD7 1, relates to a 95 kDa type II membrane glycoprotein that is present as a disulfide-linked homodimer. The transferrin receptor binds to two molecules of transferrin, which is a serum iron-transport protein, and it directs the cellular uptake of iron via receptor-mediated endocytosis. The transferrin receptor is not expressed on resting leukocytes but is upregulated upon activation of lymphocytes, monocytes and macrophages. The transferrin receptor is also found on most dividing cells and on brain endothelium.

As used herein, “EGFR” refers to a member of the family of growth factor receptors that exhibit tyrosine kinase activity. Binding of epidermal growth factor (EGF) to the extracellular domain of EGFR activates the tyrosine kinase activity of the intracellular domain. This kinase in turn triggers a cascade of events that, through a signal transduction pathway ultimately leads to increased DNA expression and cellular proliferation. The EGF-EGFR complex is transported into the cell interior by endocytosis and degraded by the proteosome rather than being recycled to the cell surface.

As used herein, the term “cytokeratin” relates to a diverse group of structural intermediate filament proteins that are expressed as pairs in both keratinized and non-keratinized epithelial tissue. Cytokeratins play a critical role in differentiation and tissue specialization and function to maintain the overall structural integrity of epithelial cells. Cytokeratins have been useful markers in identifying the origin of metastatic tumors. One cytokeratin, cytokeratin 7/8, is found in endocervical cells.

As used herein, the term “image object” refers to an aggregate group of data points from a thresholded image that are in proximity to one another. Calculable parameters of an image object include its area, defined as the number of data points included within the boundaries of the object, the average of the values of the data points in the object, the ratio of the average data value to the area of the object, and other like parameters.

Identifying Abnormal or Dysplastic Cells

The present invention identifies a plurality of markers in a cervical cell sample to determine if the sample includes dysplastic or otherwise abnormal (e.g., neoplastic or cancerous) cells. In an embodiment, the present method includes identifying one or more markers for proliferating cells in a cervical sample, identifying one or more markers for non-dysplastic cells in a cervical sample, and identifying one or more markers that distinguishes cells from debris. The invention can also include identifying one or more markers for abnormal cells.

The sequence in which a cervical cell sample is analyzed to identify the markers described herein can vary as a matter of convenience. For example, it can be convenient to first identify one or markers for proliferating cells, followed by identifying one or markers for non-dysplastic cells and finally identifying one or more markers that distinguish cells from debris. It can be convenient to first distinguish cells from debris, prior to identifying proliferating cells and proliferating non-dysplastic cells.

The present invention encompasses identifying one or more markers for proliferating cells, identifying one or more markers for normal proliferating cells and identifying one or more markers for intact cells. In this, a proliferating cell can be a cell that is proliferating as a result of normal cellular activity or as a result of dysplasia or other cellular abnormality that may or may not progress into a cancerous cell. A normal proliferating cells is a cell that is proliferating as a result of normal cellular activity. Thus, proliferating cells can encompass both healthy proliferating cells and unhealthy proliferating cells while normal proliferating cells encompass only healthy proliferating cells.

In an embodiment, the present invention encompasses identifying one or more markers for proliferating cells by treating a cell sample with a reagent that specifically, differentially or preferentially labels one or more markers for proliferating cells. One or more markers for non-dysplastic or normal proliferating cells can be identified by treating the cell sample with a reagent that specifically, differentially or preferentially labels one or more markers for non-dysplastic or normal proliferating cells. One or more markers that distinguish cells from non-cellular material and other debris can be identified by treating a cell sample with a reagent that specifically, differentially or preferentially labels one or more markers that identify intact cells or a portion thereof, or that specifically, differentially or preferentially labels one or more markers that identify non-cellular material.

In an embodiment, the present invention encompasses identifying a cellular constituent that differs in concentration between normal cells and abnormal cells. The cellular constituent that differs in concentration can be a cellular constituent that increases in concentration when a cell progresses from normal to abnormal. The cellular constituent that differs in concentration can be a cellular constituent that decreases in concentration when a cell progresses from normal to abnormal. The cellular constituent can be non-existent in one of normal cells and abnormal cells.

Cells that are not of interest can be identified and can be excluded from further consideration. The cells that are not of interest can include cells that are capable of expressing the cellular constituent that differs in concentration between normal cells and abnormal cells. Non-cellular material and other debris can also be identified and excluded from consideration.

In an embodiment, the method includes identifying one or more markers for non-dysplastic cells in a cervical sample, identifying one or more markers for proliferating cells in a cervical sample, and or identifying one or more markers that distinguishes cells from debris. It should be noted that there is no significance to the order in which these steps are presented herein, as they can be carried out in any particular order as discussed above.

Identifying Proliferating Cells

The present method can include identifying a marker or markers for proliferating cells in a cervical sample. A variety of markers for proliferating cervical cells are known and can be employed in the present method. For example, markers for proliferating cervical cells can include epidermal growth factor receptor (EGFR), transferrin receptor, and glyceraldehyde-3-phosphate dehydrogenase (GAPDH). Markers for proliferating cervical cells can include EGFR and transferrin receptor.

In an embodiment, both EGFR and transferrin receptor can be employed as markers for proliferating cervical cells. As neither EGFR nor TfR exhibit any significant intrinsic contrast relative to their surroundings on the cell membrane, an extrinsic contrast enhancing agent can be used so that these species can be discriminated from their surroundings. A reagent for identifying proliferating cells can include an antibody against EGFR or TfR (as appropriate) to which one or more (perhaps 3-10) fluorophore molecules have been conjugated. Fluorescently labeled EGF or transferrin can also be used as a ligand that will bind to either EGFR or TfR, respectively, although binding affinity and stability can be reduced.

The reagent can include a detectable label, such as a fluorescent label (e.g., fluorescein isothiocyanate (FITC) or phycoerythrin), colorimetric labels, and the like. The detectable label provides a signal that is distinct from the labels on any other reagents used. For example, if a first reagent includes a red label, the second and third reagents can include green and blue labels, respectively. Combinations of labels that are detectable in the presence of one another and whose signals can be subtracted are known and commercially available from, for example, Molecular Probes, Inc. (Eugene, Oreg.).

Chromophores can also be used as detectable labels, although fluorophores can permit adjustments in sensitivity and dynamic range by varying the integration time of the detector. Fluorophores used in the present invention can be selected to have emissions that can be resolved from cellular autofluorescence. As cellular autofluorescence typically occurs in the blue and green spectral regions, a fluorophore for an assay directed against a single cellular constituent can be selected such that emit in the red spectral region. Alexa 594 and Alexa 633 (Molecular Probes, Eugene, Oreg.) are two of many fluorophores that are suitable for this purpose.

Suitable ligands for identifying EGFR include epidermal growth factor. Antibodies (polyclonal or monoclonal) for identifying EGFR are commercially available or can be made by routine methods. Suitable anti-EGFR antibodies include those recognizing the extracellular domain of EGFR, those recognizing the cytoplasmic domain of EGFR, those raised to fresh animal cells or cell lines, those raised to native or recombinant EGFR, those with sequences found in protein or Genbank databases (e.g., with accession numbers AF329456, AF329458, or AF329457), and/or that are available from suppliers such as Chemicon International, U.S. Biological, Abcam, Santa Cruz Biotechnology, Research Diagnostics, Pharmingen, and the like.

Suitable ligands for identifying transferrin receptor include transferrin. Antibodies (polyclonal or monoclonal) for identifying transferrin receptor are commercially available or can be made by routine methods. Suitable anti-transferrin receptor antibodies include those recognizing the extracellular domain of transferrin receptor, those recognizing the cytoplasmic domain of transferrin receptor, those raised to fresh animal cells or cell lines, those raised to native or recombinant transferrin receptor, those with sequences found in protein or Genbank databases (e.g., with accession numbers AAA72800 or AAA72799), and/or that are available from suppliers such as Chemicon International, BioGenex, U.S. Biological, Abcam, and the like.

Suitable ligands for identifying GAPDH include tight binding inhibitors of this enzyme that include a detectable label. Antibodies (polyclonal or monoclonal) for identifying GAPDH are commercially available or can be made by routine methods. Suitable anti-GAPDH antibodies include those raised to native or recombinant GAPDH and/or that are available from suppliers such as Chemicon International, BioGenex, U.S. Biological, Abeam, Research Diagnostics, and the like.

Identifying Non-Dysplastic Cells

Normal cells such as normal endocervical cells can express levels of EGFR and TfR that are as high or higher than are seen in abnormal ectocervical epithelial cells, meaning that the presence of normal endocervical cells in a sample from a normal patient can cause that sample to be incorrectly classified as being abnormal. Squamous epithelial cells can be distinguished from endocervical cells on the basis of cell morphology. However, morphological differentiation is not feasible when employing flow cytometer and is difficult when using image analysis techniques. For this reason it is desirable to incorporate a reagent that discriminates between endocervical and squamous epithelial cells.

The present method can include identifying a marker or markers for non-dysplastic cervical cells. Typically, non-dysplastic cells contain this marker in an amount greater than proliferating cells. The marker for non-dysplastic cells can be present at levels not detectable in proliferating cells. A variety of markers for non-dysplastic cervical cells are known and can be used in the present method. For example, markers for non-dysplastic cervical cells include estrogen receptor, cytokeratins (e.g. cytokeratin 7, cytokeratin 8, and/or cytokeratin 13.

Examples of markers that are present in detectable amounts in non-dysplastic cells but not in abnormal cells include estrogen receptor and cytokeratins 7 and 8. In particular, cytokeratins 7 and 8 are present in high concentrations in endocervical cells, but are either not present or are present at low concentrations in squamous epithelial cells. Thus a reagent prepared by fluorescently labeling an antibody such as CAM 5.2 which specifically binds to cytokeratin 7/8 can be used to differentiate between endocervical and squamous epithelial cells. The fluorophore used to label the CAM 5.2 antibody should have a fluorescent emission at a wavelength that can be readily differentiated from the fluorescent emission from the EGFR/TfR reagent.

Identifying a marker for non-dysplastic cells can employ a reagent (e.g., a second reagent), such as a ligand or antibody, that binds preferentially to the marker. The reagent, e.g., ligand or antibody, or a molecule that binds to the ligand or antibody, includes a detectable label. Suitable detectable labels are well known and include fluorescent labels (e.g., fluorescein isothiocyanate (FITC) and phycoerythrin), colorimetric labels, and the like. The detectable label on this reagent provides a signal that is distinct from the labels on the other reagents used in the method.

Antibodies (polyclonal or monoclonal) for identifying cytokeratins (e.g. cytokeratin 7, cytokeratin 8, and/or cytokeratin 13) are commercially available or can be made by routine methods. Suitable anti-cytokeratin (e.g. anti-cytokeratin 7, anti-cytokeratin 8, and/or anti-cytokeratin 13) antibodies include those recognizing those raised to fresh animal cells or cell lines, those raised to native or recombinant cytokeratins (e.g. cytokeratin 7, cytokeratin 8, and/or cytokeratin 13), and/or those that are available from suppliers such as Chemicon International, BioGenex, Abcam, Research Diagnostics, and the like. Commercially available antibodies can recognize one or more cytokeratins, for example, antibodies that recognize human cytokeratin 13 with minor affinity for cytokeratins 14 and 17, that recognize cytokeratins 4, 5, 6, 8, 10, 13, and 18, that recognize cytokeratin 5 and/or 8, that recognize cytokeratin 7, that recognize human cytokeratin 8, that recognize cytokeratin 8 and/or 18, and the like.

Antibodies (polyclonal or monoclonal) for identifying estrogen receptor are commercially available or can be made by routine methods. Suitable anti-estrogen receptor antibodies include those recognizing the extracellular domain of estrogen receptor, those recognizing the cytoplasmic domain of estrogen receptor, those raised to fresh animal cells or cell lines, those raised to native or recombinant estrogen receptor, and/or that are available from suppliers such as Chemicon International, BioGenex, U.S. Biological, Abcam, and the like.

Identifying Abnormal Cells

The present method can include identifying a marker or markers for abnormal cells in a cervical sample. Typically, abnormal cells contain this marker at an amount greater than non-dysplastic cells. A variety of markers for abnormal cervical cells are known and can be used in the present method. For example, markers for abnormal cervical cells include progesterone receptor, cadherins, catenins (e.g., B-catenin, cyclins (e.g., cyclin E or cyclin A)), and cdk2.

Identifying a marker for abnormal cells can employ a reagent (e.g., a fifth reagent), such as a ligand or antibody, that binds preferentially to the marker. The reagent, e.g., ligand or antibody, or a molecule that binds to the ligand or antibody, includes a detectable label. Suitable detectable labels are well known and include fluorescent labels (e.g., fluorescein isothiocyanate (FITC) and phycoerythrin), colorimetric labels, and the like. The detectable label on this reagent provides a signal that is distinct from the labels on the other reagents used in the method.

Antibodies (polyclonal or monoclonal) for identifying progesterone receptor are commercially available or can be made by routine methods. Suitable anti-progesterone receptor antibodies include those recognizing the extracellular domain of progesterone receptor, those recognizing the cytoplasmic domain of progesterone receptor, those raised to fresh animal cells or cell lines, those raised to native or recombinant progesterone receptor, and/or that are available from suppliers such as Chemicon International, BioGenex, U.S. Biological, Abcam, and the like.

Antibodies (polyclonal or monoclonal) for identifying cadherins are commercially available or can be made by routine methods. Suitable anti-cadherin antibodies include those raised to fresh animal cells or cell lines, those raised to native or recombinant cadherins, and/or that are available from suppliers such as Chemicon International, BioGenex, Research Diagnostics, Abcam, and the like.

Antibodies (polyclonal or monoclonal) for identifying catenins (e.g., B-catenin) are commercially available or can be made by routine methods. Suitable anti-catenin antibodies (e.g., anti-B-catenin antibodies) include those raised to fresh animal cells or cell lines, those raised to native or recombinant catenins, and/or that are available from suppliers such as Chemicon International, BioGenex, Research Diagnostics, Abcam, and the like.

Antibodies (polyclonal or monoclonal) for identifying cyclins (e.g., cyclin E or cyclin A) are commercially available or can be made by routine methods. Suitable anti-cyclin antibodies (e.g., anti-cyclin E antibodies and anti-cyclin A antibodies) include those recognizing those raised to fresh animal cells or cell lines, those raised to native or recombinant cyclins (e.g., cyclin E or cyclin A), and/or that are available from suppliers such as Chemicon International, Research Diagnostics, Abcam, Zymed, and the like.

Antibodies (polyclonal or monoclonal) for identifying cdk2 are commercially available or can be made by routine methods. Suitable anti-cdk2 antibodies include those recognizing those raised to fresh animal cells or cell lines, those raised to native or recombinant cdk2, and/or that are available from suppliers such as Chemicon International, Research Diagnostics, Abcam, Zymed, and the like.

Distinguishing Cells From Debris

Many cervical cytology cell samples contain significant quantities of extraneous matter such as mucus, red blood cells, cell debris, bacteria and the like. Some of this extraneous material can specifically or non-specifically bind one or more of the fluorescent reagents. A portion of this discrimination can be implemented during the collection and preparation of the sample. For example, many cell preservative solutions contain mucolytic and hemolytic agents that lyse, dissolve and/or disperse extraneous matter such as mucus and red blood cells. Furthermore, common specimen preparation methods such as the “Prep” procedure from TriPath Imaging (Burlington, N.C.) include one or more steps that remove such extraneous materials from the sample.

Although useful, the available methods for the removal of extraneous materials from cervical cytology samples are frequently not 100% effective. It is therefore desirable to incorporate a means of discriminating between cells of interest and extraneous matter in the present invention. The present method can include identifying a marker that distinguishes cells from debris. This marker can be present in cells in an amount greater than in debris or present in debris in an amount greater than in cells. The marker that distinguishes cells from debris can be present at levels not detectable in debris or present at levels not detectable in cells.

A variety of markers that distinguish cells from cell or tissue debris are known and can be used in the present method. Suitable markers that distinguish cells from cell or tissue debris include DNA, as the cells of interest are nucleated and contain DNA whereas most types of extraneous matter encountered in cervical cytology samples do not contain DNA and those types of extraneous matter (such as bacteria) that do contain DNA do so in nuclei that are much smaller than those in the cells of interest. Numerous fluorescent DNA stains are known in the art. One major selection constraint is that it should be possible to easily discriminate the fluorescent emissions of the stain selected from those of the other constituents of the assay.

Suitable reagents (e.g., a third reagent) for detecting DNA are known and include the DNA minor groove binding dye 4,6-diamido-2-phenylindole, dihydrochloride (DAPI), which predominately binds to intact, double stranded DNA, and fluoresces blue, acridine (green/red), acridine orange (red), 7-aminoactinomycin D (red), ACMA (blue), dihydroethidium (red), ethidium bromide (red), ethidium homodimer-1 (red), ethidium homodimer-2 (red), ethidium monoazide (red), hexidium iodide (red), Hoechst 33258 (blue), Hoechst 33342 (blue), Hoechst 34580 (blue), propidium iodide (red), and more recently developed known DNA stains, and the like.

TO-PRO-3 is representative of a number of fluorescent DNA stains that are quantitative in that the level of fluorescence produced is proportional to the amount of DNA present. If such a quantitative DNA stain is employed, the integrated signal intensity of nuclear objects identified by segmentation represents the chromosome number or “ploidy” of the cell. Ploidy is increased in cells that are undergoing cell division and in cells that have undergone malignant transformation. In normal cell division, the chromosome number cycles between one and two sets of chromosomes per cell depending upon where the cell is in the division process. Transformed cells can, however, have three or more sets or fractional sets of chromosomes. Thus ploidy can be used as another parameter in the cell classification procedures described herein.

Identifying Dysplastic Cells

Material identified as proliferating cells can include both cells and debris. Excluding or subtracting the debris from this material yields net proliferating cells. Cells identified as proliferating include both dysplastic and non-dysplastic proliferating cells. Excluding or subtracting proliferating, non-dysplastic cells from the identified (net) proliferating cells yields those cells that are dysplastic. Subtraction is commutative and can occur in any order that yields the dysplastic cells.

Material identified as abnormal cells can include both cells and debris. Excluding or subtracting the debris from this material yields net abnormal cells. Cells identified as abnormal can include both dysplastic and non-dysplastic cells. Excluding or subtracting non-dysplastic cells from the (net) identified abnormal cells yields those cells that are dysplastic. Subtraction is commutative and can occur in any order that yields the dysplastic cells.

Cocktail for Identifying Dysplastic Cells

The cocktail of the present invention includes reagents that recognize a plurality of markers in a cervical cell sample for determining whether the sample includes dysplastic (e.g., neoplastic or cancerous) or otherwise abnormal cells. The present cocktail includes one or more reagents that recognize one or more markers for proliferating cells, one or more reagents that recognize one or more markers for non-dysplastic or normal proliferating cells, and one or more reagents that recognize one or more markers that distinguish cells from debris.

In an embodiment, the cocktail includes one or more reagents that recognize one or more markers for non-dysplastic cells or normal proliferating cells, one or more reagents that recognize one or more markers for proliferating cells in a cervical sample, and one or more reagents that recognize one or more markers that distinguish cells from debris.

Preferred cocktails include combinations of reagents that, for example, when their corresponding detectable signals are subtracted, yield identification of dysplastic cells. In particular, the present cocktail provides for rapidly and accurately identifying abnormal cells while discounting false positives from otherwise normal cells (such as endocervical cells) and cellular debris.

In an embodiment, such a cocktail includes one or more reagents (e.g., a first reagent) that recognizes proliferating cells, one or more reagents (e.g., a second reagent) that recognizes non-dysplastic proliferating cells, and one or more reagents (e.g., a third reagent) distinguishing cells from debris. It has been determined that this preferred combination of reagents is surprisingly effective in effectively identifying abnormal cells in a cervical cell sample.

In an embodiment, such a cocktail includes one or more reagents (e.g., a fourth reagent) that recognizes abnormal cells, one or more reagents (e.g., a second reagent) that recognizes non-dysplastic proliferating cells, and one or more reagents (e.g., a third reagent) distinguishing cells from debris.

The reagents making up the cocktail can be provided as one of more distinct compositions. For example, the cocktail can be provided as separate containers of each of reagent together with instructions for mixing the reagents or applying them to a cell sample. In an embodiment, these separate reagents can be mixed by the user before use to provide a complete cocktail including all of the reagents. Alternatively, the cocktail can be provided as a single composition including each of the reagents. By way of further example, one or more of the reagents can be in a single composition with one or more reagents separate from that composition. This can be desirable when certain reagents are incompatible for storage or shipping.

Image Analysis System and Method

A variety of image processing or analysis systems can be employed in the present method, with the present cocktail, and as a part of the present system. The image analysis system typically includes a mechanism for obtaining an image, such as an array of charge coupled devices. The image analysis system also includes one or more processors adapted and configured for identifying a labeled dysplastic or otherwise abnormal cell in a cell sample, such as a cervical cell sample.

The processor identifies the dysplastic cell or cells by detecting one or more markers for proliferating cells in a cervical sample, detecting one or more markers for non-dysplastic cells in a cervical sample, detecting one or more markers for abnormal cells in a cervical sample, and/or detecting one or more markers that distinguishes cells from debris.

The material detected as proliferating cells can include both cells and debris. The system processor excludes or subtracts the detected debris from material detected as proliferating cells to yield net proliferating cells. The net proliferating cells include both dysplastic and non-dysplastic proliferating cells. The system processor excludes or subtracts the detected proliferating, non-dysplastic cells from the net proliferating cells to yield those cells that are dysplastic. Subtraction is commutative and can occur in any order that yields the dysplastic cells.

The material detected as abnormal cells can include both cells and debris. The system processor excludes or subtracts the detected debris from material detected as abnormal cells to yield net abnormal cells. The net abnormal cells include both dysplastic and non-dysplastic cells. The system processor excludes or subtracts the detected non-dysplastic cells from the net abnormal cells to yield those cells that are dysplastic. Subtraction is commutative and can occur in any order that yields the dysplastic cells.

In an embodiment, the processor identifies the dysplastic cell or cells by detecting one or more markers for non-dysplastic cells in a cervical sample, detecting one or more markers for proliferating cells in a cervical sample, and/or detecting one or more markers that distinguishes cells from debris. In this embodiment, the processor can exclude or subtract detected debris from material detected as proliferating cells to yield net proliferating cells. In this embodiment, the system processor can exclude or subtract the detected proliferating, non-dysplastic cells from the net proliferating cells to yield those cells that are dysplastic. Subtraction is commutative and can occur in any order that yields the dysplastic cells.

As a practical matter, the concentration of DNA in a cell far exceeds the concentrations of any of the other species (EGFR, TfR, cytokeratin, etc.) of interest. Furthermore, many fluorescent DNA stains have relatively broad spectral emissions and therefore can interfere with measuring fluorescent emissions from the other fluorophores. For example, if the “blue” fluorophore DAPI is used as the DNA stain and the “green” fluorophore fluorescein is used to label one of the other assay components, the contribution of DAPI fluorescence to the signal measured at the fluorescein emission wavelength will be about half of the signal level measured at the wavelength of maximum DAPI emission.

One way to address this issue is to calibrate the detection system such that the contribution of each fluorophore, most conveniently expressed as a fraction of the peak emission intensity for the fluorophore, to each detection channel is accurately known. When a measurement is made of an actual patient sample, the measured signal level in each detection channel will reflect the sum of the contributions of each of the fluorophores to that channel. If the number of detection channels equals or exceeds the number of fluorophores, the measurement and calibration data can be combined to form a set of simultaneous equations that can be solved using standard methods to yield the actual contributions of each fluorophore to each detection channel.

Another approach is to select the fluorophores such that the DNA stain has the longest emission wavelength and does not significantly overlap the next shorter detection wavelength band. This effectively isolates the EGFR/TfR (for example) and CAM 5.2 (for example) reagent emissions from those of the DNA stain. This practice can be extended to assigning the fluorophore used in the EGFR/TfR reagent to the shortest wavelength channel so that it is not overlapped by any of the other fluorophores in the assay reagents.

For the purposes of the following non-limiting discussion, it will be assumed that the assay configuration will include three contrast enhancement reagents, one each specific for EGFR or TfR; cytokeratin 7/8 and DNA; that the fluorophores associated with these reagents are Alexa 594 (red); fluorescein (green) and DAPI (blue), respectively; and that the detection device incorporates detection channels corresponding to each of these three fluorophores.

If a flow cytometer is used as the detection device, an additional detection channel that measures the amount of light scattered from each object passing the detection point can also be present. This channel has utility in the estimation of the size of each cell; as a coincidence trigger for data acquisition; and as a range gate for the inclusion or exclusion of cells from the population to be evaluated.

In an embodiment in which a flow cytometer is used as the detection method, the appearance of a peak in the output of the light scatter detector denotes the presence of an object in the light path while the amplitude and width of the pulse can be used as empirically determined selection criteria to determine whether or not the object in the light path is a cell of potential interest.

If it is determined based upon the light scattering characteristics that the object in the object in the light path is a cell of potential interest, the outputs of the red, green and blue detection channels temporally coincident with the scatter peak are captured for further analysis. The data captured from the red, green and blue can be compared against selection criteria determined during the calibration of the cytometer in order to classify the object.

In the particular assay configuration assumed for the purposes of this description where the DNA stain is assigned to the blue detection channel, the spectral emissions from the DNA stain can overlap into the green and red detection channels. In this instance it is desirable to correct the data captured in the green and blue detection channels for the presence of this spectral overlap prior to performing object classification. This spectral overlap correction, which may also pertain to signal from the green fluorophore overlapping into the red detection channel, can be performed by the method described in a previous paragraph.

Object classification can be performed on the basis of comparison of the (optionally corrected) data against a series of selection criteria. If, for example, the output of the blue detection channel is greater than a predetermined threshold thus denoting the presence of a nucleated cell and the width of the detection peak in the blue channel is greater than the value corresponding to the smallest nuclear size of interest and the output of the red detection channel is greater than the threshold corresponding to a higher than normal cell surface concentration of EGFR/TfR, but the output of the green detection channel is less than the threshold corresponding to the concentration of cytokeratin 7/8 in an endocervical cell then the object is classified as being an abnormal squamous epithelial cell.

Similar selection criteria can be established for the classification of other object types. Although the decisions leading to such a classification can be performed sequentially or in a decision tree format, the use of a multivariate classifier is to be preferred as it intrinsically accommodates parametric interactions.

The preceding description of object classification implies that object classification is performed in the red-green-blue (RGB) color space. Depending upon the specific fluorophores utilized in the assay and the relative concentrations of each of the species being detected, it is sometimes beneficial to transform the data from the RGB color space into an alternative color space such as hue-saturation-luminance (HSL) prior to classification or to incorporate both RGB and alternative color space representations of the data in the classification. Operation in an alternative color space is of particular utility in those cases where there is significant spatial and spectral overlap between objects of the different fluorescent colors.

In those cases where the sample is presented on a planar surface such as a microscope slide, an array-type detection device such as a linear or area CCD array can be used to capture the necessary images. In those cases where the cellular samples are presented on a non-planar surface such as those of a cell collection device, a single point device such as a flying spot scanner can be used.

For the purposes of the following descriptions it will be assumed that the specimen is presented on a microscope slide and that the image capture device is a color CCD camera that provides spatially resolved area array images in the red, green and blue spectral bands. It will further be assumed that the camera is mounted on a motorized microscope platform that permits the specimen slide to be positioned and the images focused under computer control. Similar considerations will apply to alternative implementations such as those utilizing line scan and flying spot detection devices. As in the case of the flow cytometer embodiment described previously, the following description will implicitly assume a RGB color space although HSL and other alternative color spaces can be beneficial in many instances.

Images captured through a microscope using a CCD area array camera frequently suffer from a variety of non-idealities such as a falloff of signal intensity toward the periphery of the image, “hot” and “dead” pixels in the CCD array(s), and the like. In order to facilitate subsequent analysis it is desirable to correct each captured image for any such non-idealities prior to classifying the objects present in the image. Methods for performing such “shading” corrections are well known. It is also desirable to correct for spectral overlap of the fluorophore emissions into multiple detection channels before classifying the objects. These corrections can be performed for each pixel in the image by the methods described previously.

Due to the substantial difference in signal level in the blue (DNA in this example) channel on one hand and the red and green channels on the other, it is sometimes convenient to rescale the blue signal levels such that they are comparable to those in the red and green channels. One convenient means of performing this resealing is to capture two successive images of the same field of view. One of these images is captured at an exposure time that is long enough to place the red and green image data roughly in the middle of the dynamic range of the camera.

Similarly, the second image is captured at an exposure time that is short enough to ensure that the blue image pixels are not saturated. So long as the blue pixels are not saturated, the blue channel data from the shorter of the two exposures can be used to compute the spectral overlap corrections due to the emissions from the blue fluorophore overlapping into the green and red detection channels. In some instances, the short exposure image is also of use in computing the corresponding corrections for the overlap of the emissions of the green fluorophore into the red channel as either the green signal is saturating or the spectral overlap from the blue fluorophore plus the true green signal is sufficient to saturate the pixel.

Performing the blue channel segmentations on the image data from the shorter of the two exposures also ensures that these segmentations are performed on a consistent basis such as a percentage of full height, something that is not possible when a significant number of pixels in the image are saturated.

The mechanics of the classification of the various objects in these images is very similar to that described previously with respect to the flow cytometer embodiment of this invention. The major point of difference is that the analysis of the flow cytometer data deals with discrete events while image data analysis deals with the spatial distribution of the various fluorophores on and within the individual cells. For this reason it is desirable to segment the image data into discrete groups of pixels, each group corresponding to a cell, and to treat these groups of pixels as objects during the subsequent analysis and classification. Two general approaches have been utilized in the present invention for the assignment of image pixels to specific objects.

One approach is based upon the fact that only nucleated (i.e., DNA containing) cells are of potential interest in the screening of a specimen. In this approach, the blue (DNA) image is thresholded to define clusters of pixels that correspond to individual putative cell nuclei. Standard techniques such as dilation and erosion can then be used to smooth and refine the boundaries of these putative nuclei. Standard metrics such as area and axial ratio can be computed for each of these objects and used to select only those objects for which the metrics are consistent with those of nuclei known to be of interest for further analysis. Each of the blue objects identified as being a cell nucleus is then used as a center to which red and green pixels in the vicinity of the center are allocated.

One simple means of allocating surrounding pixels to various nuclear centers is to allocate all pixels within a pre-determined radius of the center to that nucleus. A variant on this procedure is to define an annulus having predefined characteristics such as width around each nuclear center and to allocate all pixels falling within an annulus to the corresponding nucleus. Methods such as these are fast and efficient if the separation between nuclear centers is greater than the sum of the radii of the zones drawn around adjacent centers. In those cases where these zones overlap, additional criteria such as “nearest neighbor” can be applied to ensure that each pixel is associated with one and only one nucleus. More computationally intensive methods such as “region growing”, “principle components analysis”, “K-means” and “watershed” can also be used to allocate pixels to the appropriate nuclear centers and are generally desirable, if not necessary, when the cells on the slide are not well dispersed.

An alternative approach to pixel assignment is based upon the fact that only objects that exhibit the color of fluorescence (red in this example) associated with cellular abnormality are of interest in the analysis. In this approach, the red, green and blue images of a given field of view are individually segmented using a thresholding or similar operation to group the image pixels into objects. A mathematical “union-of-sets” operation is then used to associate the objects identified in the blue and green image planes with objects identified in the red image plane. Again, this method is most effective when the cells on the slide are well dispersed and more computationally intensive methods such as identified above may be necessary to complete the pixel allocation process for cases where, for example, multiple blue objects are found to be embedded within a single red object. The segmentation of the image(s) into objects is often more conveniently and simply accomplished in HSL color space than in RGB color space, especially when spectral overlap correction has not been performed or the quality of the correction is uncertain.

Once pixel allocation has been completed, the identified objects can be classified using a multivariate classifier such as described in conjunction with the flow cytometer embodiment of this invention.

Once the cells in a specimen have been classified using either the flow cytometer or image analysis method described above, it is preferable to assign a classification to the entire sample. In many cases, particularly in the US, it is desirable to initially assume that every sample is abnormal and then to reclassify as normal only those samples that show no signs of abnormality. This “select out” approach ensures that even borderline cases are identified for further evaluation. The alternative is a “select in” approach in which all samples are initially assumed to be normal and only those exhibiting signs of abnormality are reclassified as being abnormal. Both approaches include procedural controls to ensure that the sample preparation, staining and data capture have been performed correctly and that the data meet certain minimum criteria for acceptability.

One method of sample classification is to adopt an absolute criterion such as the identification of one (or some other predetermined number of) abnormal objects in a sample is sufficient to cause the sample to be classified as being abnormal. While being easy to implement, this type of absolute method is “brittle” in that is very sensitive to parameters such as sample characteristics, procedural variations and the number of objects included in the analysis. A more robust method is to accumulate object classification data from a large number of specimens that have been classified using an independent procedure such as morphological analysis; subject the resulting data to a statistical distribution analysis; and defining a decision threshold based upon a statistical parameter such as the standard deviation of the distribution of normal cell measurements that, on a statistical basis, can be predicted to result in an acceptable level of assay sensitivity and specificity.

Particularly when assays such as the present invention are employed in a screening environment, assay throughput in terms of the turnaround time for a sample or the number of samples that can be processed within a given period of time is a major consideration. Many aspects of improving throughput such as the use of faster computers and communications links; optimizing assay chemistries and analytical software algorithms and the like are well known and are applicable to the present invention. Similarly, “tricks” for improving system throughput such as using a high numeric aperture objective lens in conjunction with an intermediate magnification reducing lens to enlarge the field of view of a microscope while maintaining a high light capture efficiency are also well known.

Another method for improving throughput is to capture data from only the minimum number of cellular objects required to reliably classify the specimen. Classifying a specimen as being abnormal requires the detection of only one or a small number of abnormal cells which means that data capture and analysis can safely be terminated as soon as this threshold number of events has been reached. The strict criterion for declaring a specimen to be normal is that no abnormal cells be detected during the examination of every cell in the specimen. Adhering to this criterion can substantially reduce throughput as a typical cervical cytology can contain tens to hundreds of thousands of cells.

An alternative method is to determine the number of cells that need to be evaluated in order to have a predetermined degree of confidence that the classification of the specimen as normal is correct. This number can be computed using a statistical “power” calculation such as is used in determining the number of patients needed in a clinical trial. Limiting the number of cells to be evaluated in this way improves throughput for highly cellular samples; ensures that at least the minimum amount of data required to deliver a result having the desired confidence level; and provides a standardized condition that facilitates comparison of results from specimens having a wide range of cellularities.

Two additional benefits accrue from the present invention. By international convention and, in some countries, by law, a cervical cytology specimen should contain a certain minimum total number of cells and a certain minimum number of endocervical (and/or metaplastic) cells in order for the specimen to be considered to be “adequate” for screening and diagnosis. Both the flow cytometer and imaging based embodiments of the present invention intrinsically provide this information based upon the signals produced by the second and third reagents thus ensuring that results are reported only for “adequate” specimens.

The second benefit is realizable primarily in the imaging based embodiment of the present invention, particularly, but not exclusively the format in which the specimen is presented on a microscope slide. As previously cited, the analytical reagents and procedures used in this invention do not affect cell morphology or the ability of the cells to be stained with chromatic chemical stains. Thus, after the specimen has been stained with the fluorescent reagents of this invention and the corresponding data captured; the same specimen can be counterstained with a chromatic reagent such as PAP stain and then visually examined for the purpose of assessing the morphologies of those cells that were declared to be abnormal on the basis of fluorescence staining. This provides the ability to rapidly, conveniently and independently confirm the results delivered by the present invention without the need to collect or prepare an additional sample.

As previously stated, the above descriptions of the embodiments of the present invention assumed that the DNA stain had the shortest wavelength of fluorescent emission while the EGFR/TfR reagent had the longest emission wavelength. This assumption was made in order to better illustrate certain aspects of the present invention. Significant operational improvements can be made by selecting/constructing the reagents such that the reagent for the analyte that is present in the highest concentration (DNA) has the longest emission wavelength and the reagent for the analyte that provides discrimination between normal and abnormal cells (EGFR and/or TfR) has the shortest emission wavelength.

This revised ordering eliminates the possibility of the strong emissions from the DNA reagent or the potentially strong emissions from the CAM 5.2 reagent from overlapping into the detector channel associated with the EGFR/TfR reagent. In turn, this minimizes and possibly eliminates the need for the previously described spectral overlap corrections and the potential errors associated therewith, thus reducing the time and computing power needed to process the captured data. One suitable set of fluorophores for use in this revised arrangement consists of“BODIPY” as the fluorophore for the EGFR/TfR reagent; fluorescein or Alexa 488 or 532 as the fluorophore for the CAM 5.2 reagent; and TO-PRO-3 as the DNA stain. These fluorophores are available from Molecular Probes (Eugene, Oreg).

By way of example, consider an embodiment in which each of the reagents used in the cocktail and method binds to a specific marker. In this embodiment, a first reagent or reagents are used that bind to markers that are preferentially expressed by abnormally proliferating cells. The reagent or reagents can be tagged with a detectable label, such as a fluorophore, of a particular color. For purposes of illustration, this first reagent is tagged with a fluorophore that expresses the color red.

Because endocervical cells are frequently proliferative, they can express some of the same markers as abnormally proliferating cells. Thus, the presence of endocervical cells can provide a false positive unless otherwise accounted for. In this illustrative embodiment, a second reagent is used that preferentially binds to endocervical cells. This second reagent can be tagged with a detectable label, such as a fluorophore, of a second color. A detectable label of a second color can be subtracted from an image including the first label of a first color. For purposes of illustration, this second reagent is tagged with a fluorophore that expresses the color green.

Cell samples often contain significant amounts of debris, which can provide false positive readings unless otherwise accounted for. The method and system can optionally identify or detect a third reagent that binds to DNA and includes a detectable label. Any detected or identified material or cells that lack DNA can then subtracted or excluded. That is, any material having the color of the first and/or second reagent but not the color of the third reagent can be subtracted or excluded. For purposes of illustration, this optional marker is tagged with a fluorophore that expresses the color blue.

Thus, in an embodiment, a marker cocktail can include one or more red-tagged reagents that identifies or detects abnormally growing cells, one or more green-tagged reagents that identifies or detects endocervical cells, and a blue-tagged reagent that identifies or detects material lacking DNA. Obviously, these colors are arbitrary and any colors can be chosen as desired so long as they can be subtracted from one another.

Sample Preparation

Cellular samples such as cervical cells that are suitable for use in the present method, with the present cocktail, and in the present system can be collected and processed in a variety of ways, as described in greater detail hereinbelow. For example, cervical cells can be collected via a brush or spatula as is commonly done in the traditional PAP test. Alternatively, cervical cells can be collected using a personal collector as described for example in U.S. Pat. No. 6,352,513 which is incorporated by reference herein, or by or using a physician's collector, as described herein.

Once the cells have been collected, they can be processed in any of a variety of ways. A cell sample can be processed by conventional or other methods to provide cells suitable for examination on a microscope slide. These cells can be treated with reagents, such as the present cocktail on the slide, or before they are put on the slide. A cell suspension can also be processed to provide a representative sampling of the cells immobilized on a membrane or other substrate. These immobilized cells can be treated with the present cocktail and can be analyzed while remaining on the membrane. An image of the cells present on the membrane can be captured and processed to detect abnormal cells and to report their relative locations.

The in-vitro version of this assay can be performed on cellular samples that have been fixed or otherwise preserved to prevent cell damage and degradation. Suitable preservatives include, but are not limited to 95% ethanol containing 2-5% poly-ethyleneglycol, PreservCyt (Cytyc Corp., Boxborough, Mass.), Cytorich (AutoCyt, Burlington, N.C.) and others known to those skilled in the art. The primary constraints on the selection of the preservative are that it adequately preserve the cells of interest and that it not exhibit significant fluorescence at the emission wavelength of the fluorophore.

The cellular sample can be in the form of a cellular suspension in a preservative solution. Most cellular preservatives can have an adverse effect on immunological reactions such as are employed in this assay. Therefore, it is desirable to separate suspended cells from the preservative and to remove residual preservative from cells that have been deposited onto a surface. The former is most readily accomplished by sedimenting the cells to form a pellet, removing the resulting supernate and resuspending the cell pellet in a medium such as PBS (nominally 0.05 M phosphate, 0.15 M NaCl pH=8.0). This process can be repeated multiple times if complete removal of the preservative is desired. Residual preservative can be removed from cells deposited on a surface by immersing the surface in a medium such as PBS.

Although antibodies generally bind very specifically to the epitope(s) against which they were raised, they may also bind to cells through a variety of non-specific mechanisms. In an assay, it is desirable to suppress this non-specific binding as it reduces the contrast between the objects of interest and their surroundings. Numerous means for blocking non-specific binding such as treatment of the sample with a dilute (typically about 2%) solution of a protein such as bovine serum albumin or casein are known to those skilled in the are and are suitable for use in this assay. The mechanics of performing this blocking step can be the same as used for the removal of the preservative.

After blocking of non-specific binding sites, the sample is exposed to the selected immunoreagent (preferably fluorescently labeled anti-EGFR or anti-TfR) in the same manner as the previous steps. At the end of an empirically determined incubation time, the cells are separated from the immunoreagent and washed several times with PBS or a similar medium using methods similar to those described previously.

Final preparation of the cell sample for measurement depends upon the form of the sample and the method selected for performing the measurement. Cells in suspension generally require no further preparation and are most conveniently measured using a flow cytometer.

Cells deposited on a microscope slide are preferably coverslipped in the conventional manner using a non-fluorescent mounting medium. Specimens of this type are most conveniently measured using an automated microscope equipped with a video camera for image capture, and software for the quantitation and interpretation of the captured images. Cells on the surface of a cell collection device are most conveniently washed with 95% ethanol possibly containing 2% poly-ethyleneglycol, allowed to dry, and measured in-situ. In each case, the intensity of the fluorescence associated with each cell is a measure of the concentration of the target analyte (EGFR or TfR) on the surface of the cell.

Comparison of these measured intensities against those of populations of known normal and abnormal cells of the same types permits the classification of the individual cells and the entire sample as being normal or possibly abnormal.

Collectors

A cervical cell collector can include sampling means for sampling a cervix in a spatially resolved manner, supporting means for supporting the sampling means, and inflation means for reversibly inflating and deflating the sampling means. See for example U.S. patent application Ser. No. 09/725,332, which is incorporated by reference herein.

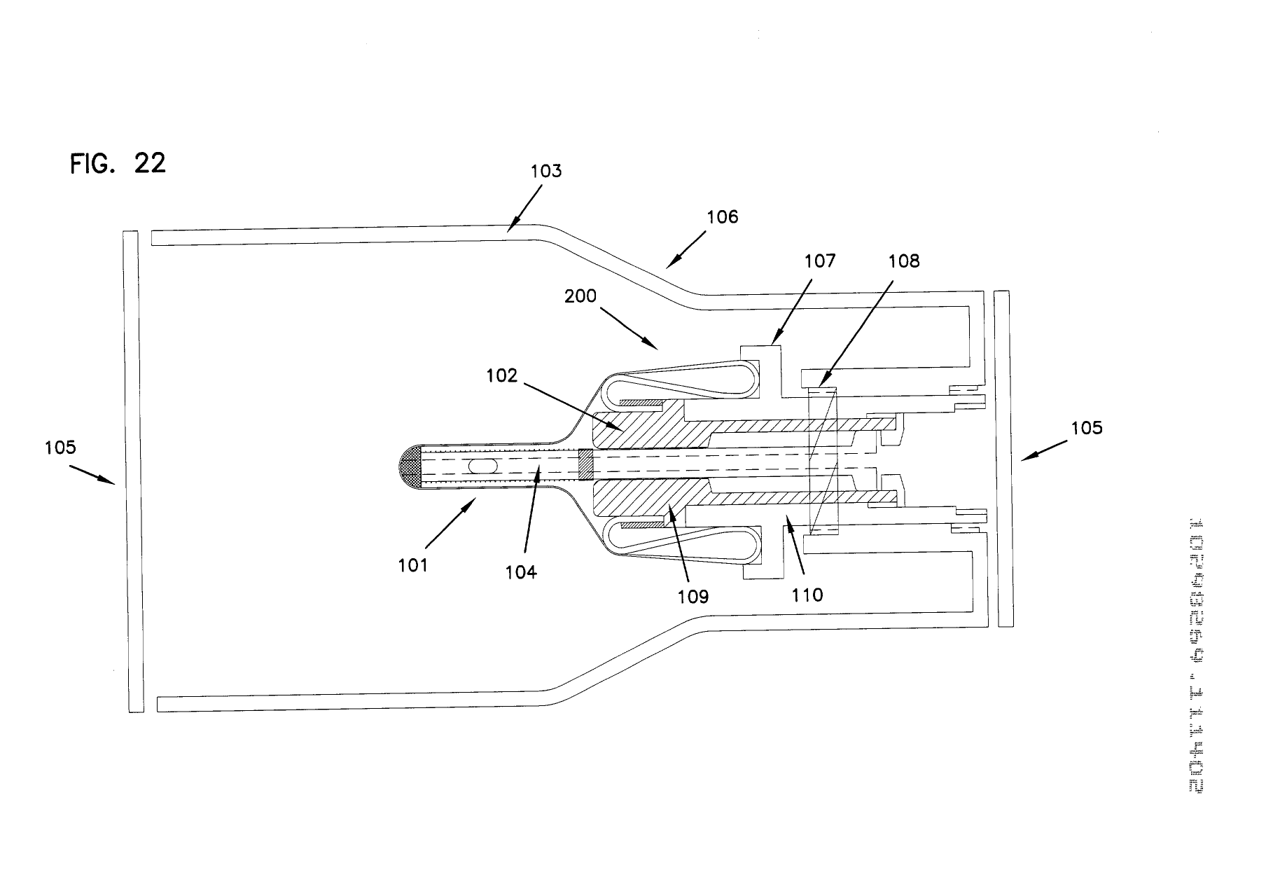

FIG. 22 shows a physician's collector that includes a compliant cell sampling member 101 that is attached to an interface element 102 and that is mounted within a multifunctional container 103. A stylet 104 passes through the interface element 102 and into the cell sampling member 101 where it is bonded to the interior of the tip of the cell sampling member 101. The sampling member 101 can be a balloon structure that is made of a suitable elastomeric material such as silicone rubber, latex rubber, polyurethane or a thermoplastic elastomer.

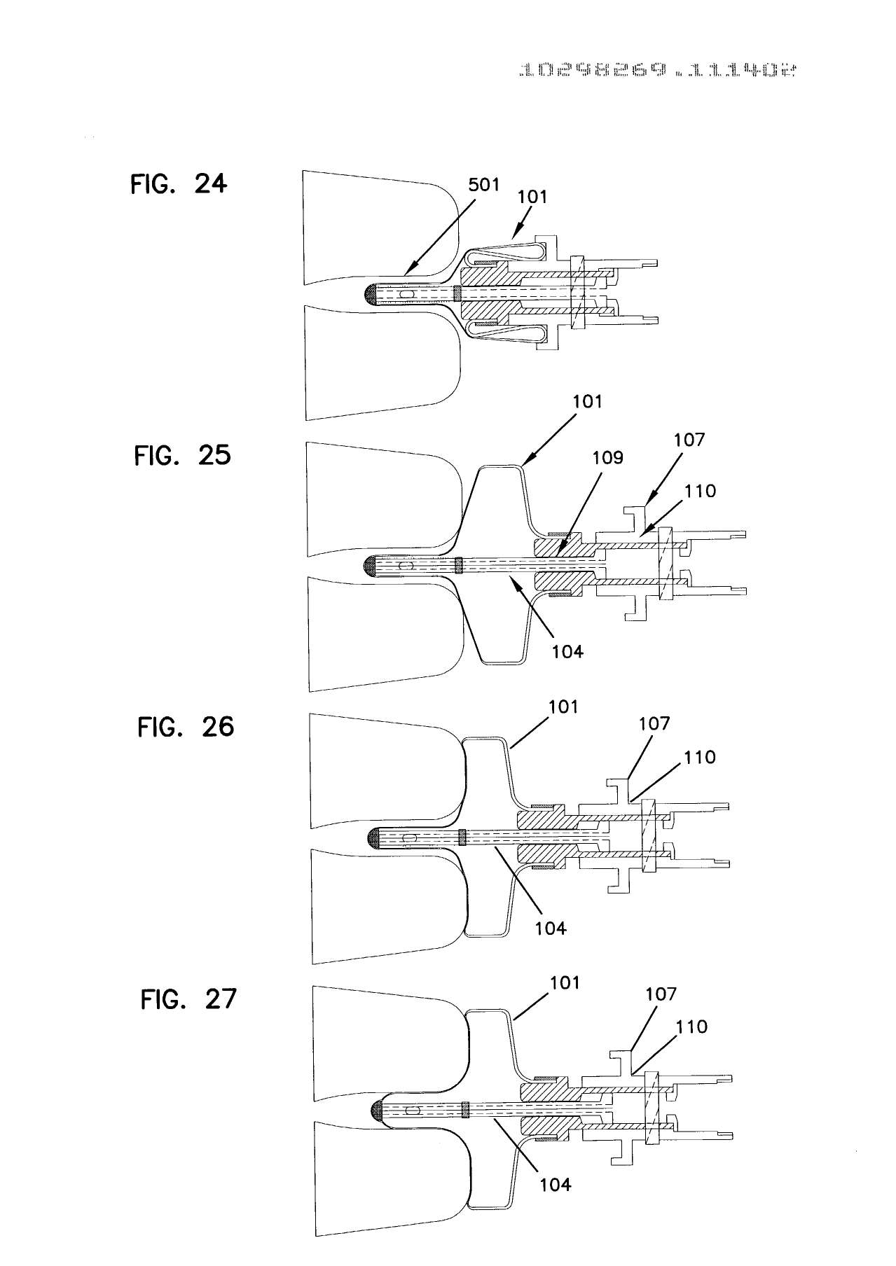

As suggested by FIGS. 24-27, the clinician guides the device through a speculum into the vagina and inserts the tip of the sampling member 101 into the cervical canal 501 until the shoulder of the sampling member 101 seats against the cervix. See FIG. 24. The flattened shape of the sampling member affords the clinician improved visibility of the cervix during insertion, while the bullet shaped tip of the sampling member and the stiffness imparted by the stylet 104 facilitate insertion of the sampling member 101 into the cervical canal 501.

Insertion of the tip of the sampling member 101 into the cervix proceeds until the body of the sampling member 101 is properly seated against the mouth of the cervix, the sampling member 101 is deployed from its initial folded state into its unfolded sampling configuration as is shown in FIG. 25.

Deployment is triggered partially by depressing the actuator button 303 at the end of the handle 301. The internal structure of the handle 301 can, in essence, be described as a syringe, the major elements of which consist of a barrel 304 that slides within the handle 301, an extension of which forms the actuator button 303; and a plunger 305 having an elongated hollow shaft 306 that is slideably retained in the body of the handle 301 and which makes air tight sliding contact with the interior of the barrel 304.

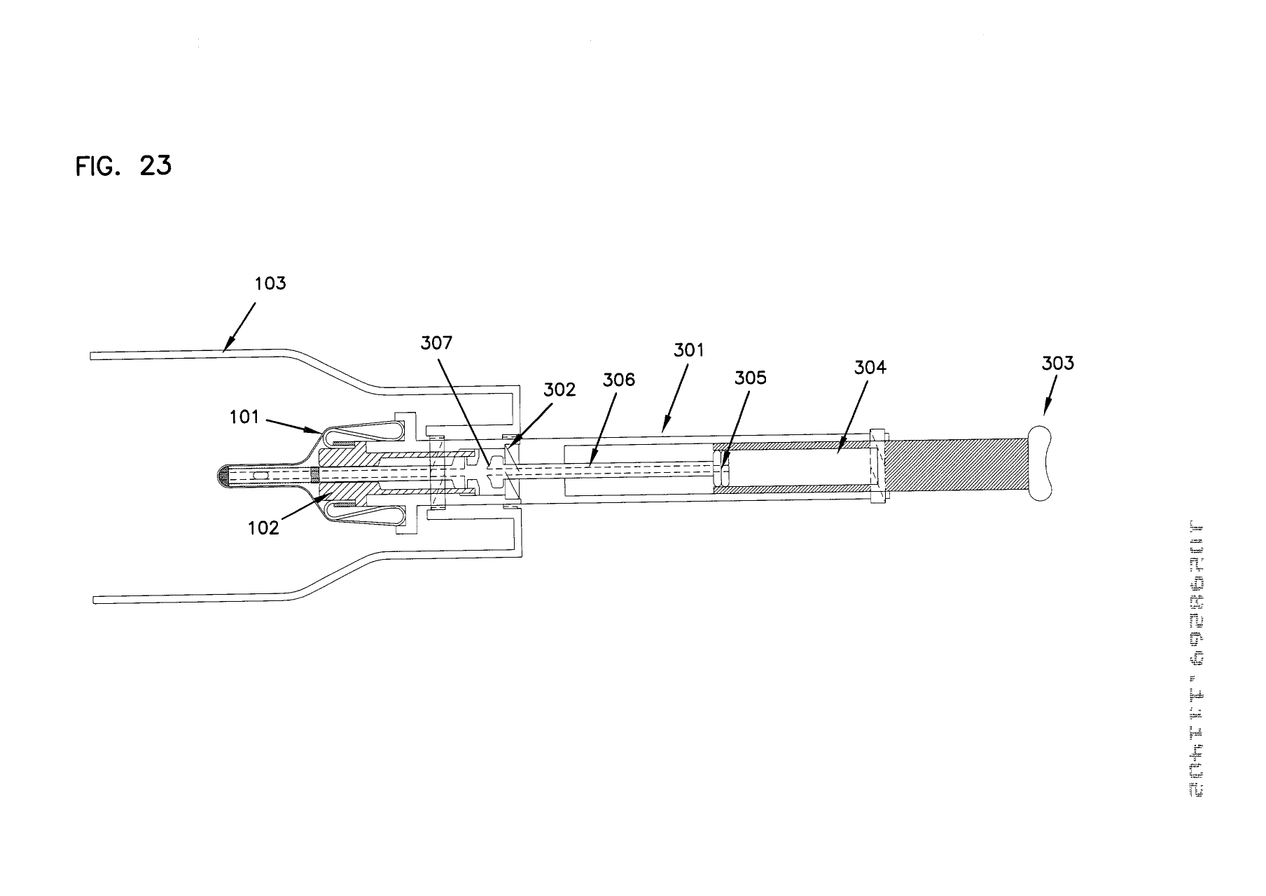

As the button 303 (FIG. 23) is initially depressed, friction between the plunger 305 and the barrel 304 causes the barrel 304, plunger 305 and hollow shaft 306 to move in unison in the direction of the sampling member 101 until the end 307 of the hollow shaft 306 makes contact with the inner portion of the interface element 103. Deployment ceases when the rear end of the stylet 104 seats against the inner surface of the inner portion of the interface element 109 forming an air tight seal. As is shown in FIGS. 26-27, pushing the handle toward the patient causes the side walls 202 of the sampling member to move toward and ultimately to contact the surface thus draping the front wall of the sampling member 203 over the cervix.

Sampling by a contact means rather than by an abrading or scraping means as is employed by other cervical cell sampling devices confers certain benefits to the clinician. Cancerous lesions initiate at discrete foci and, except in extreme cases, are localized to specific regions of the cervix at the time of sampling. Contact sampling retains the spatial or topological relationships between the collected cells. Assuming that a lesion is present on the cervix, retention of the spatial relationships results in regions on the cell collection surface that are enriched in abnormal cells.

Localization of the abnormal cells on the surface of the cell collection device results in a higher signal to noise ratio between normal and abnormal cells, and thus improved detectability of abnormal cells relative to what is possible with samples obtained by an abrasive or scraping means where in the abnormal cells are dispersed throughout the sample.

Furthermore, localized sampling in conjunction with in-situ detection on the collection surface permit the locations on the cervix of any lesions detected to be presented to the clinician in a manner that directs the clinician to these specific areas of the cervix for the purposes of clinical follow-up. Certain features of the present invention such as those that establish the orientation of the sampling member with respect to the cervix are particularly intended to support and facilitate use of the present invention in this manner.

Once the cellular sample has been collected on the surface of the sampling member 101, the device is removed from the patient. This is facilitated by releasing any air that may have been injected into the sampling member 101 during the sampling process. In the specific embodiment described here, release of the pressure being applied to the actuating button 303 allows the air pressure within the sampling member 101 to push on the syringe elements 304, 305, 306 within the handle 201 so that the entire syringe element moves away from the sampling element 101. This releases the mechanical pressure that maintains the air tight seal 601 between the hollow shaft of the plunger 306 and the interface element 109. Breaking these seals allows the escape of any air pressure within the sampling member that was not relieved by motion of the syringe element.

Instrument Analysis on Collector

A cellular sample can be evaluated in a manner in which the spatial relationships existing between said cells in-vivo are preserved and can be used to guide subsequent follow-up and treatment where abnormalities are detected. Cervical cells can be collected on a non-planar surface, wherein the cervical cells can be arranged on the non-planar surface in accordance with their orientation prior to collection and can be stained and analyzed while remaining on the non-planar surface. See for example U.S. patent application Ser. No. 09/725,795, which is incorporated by reference herein.

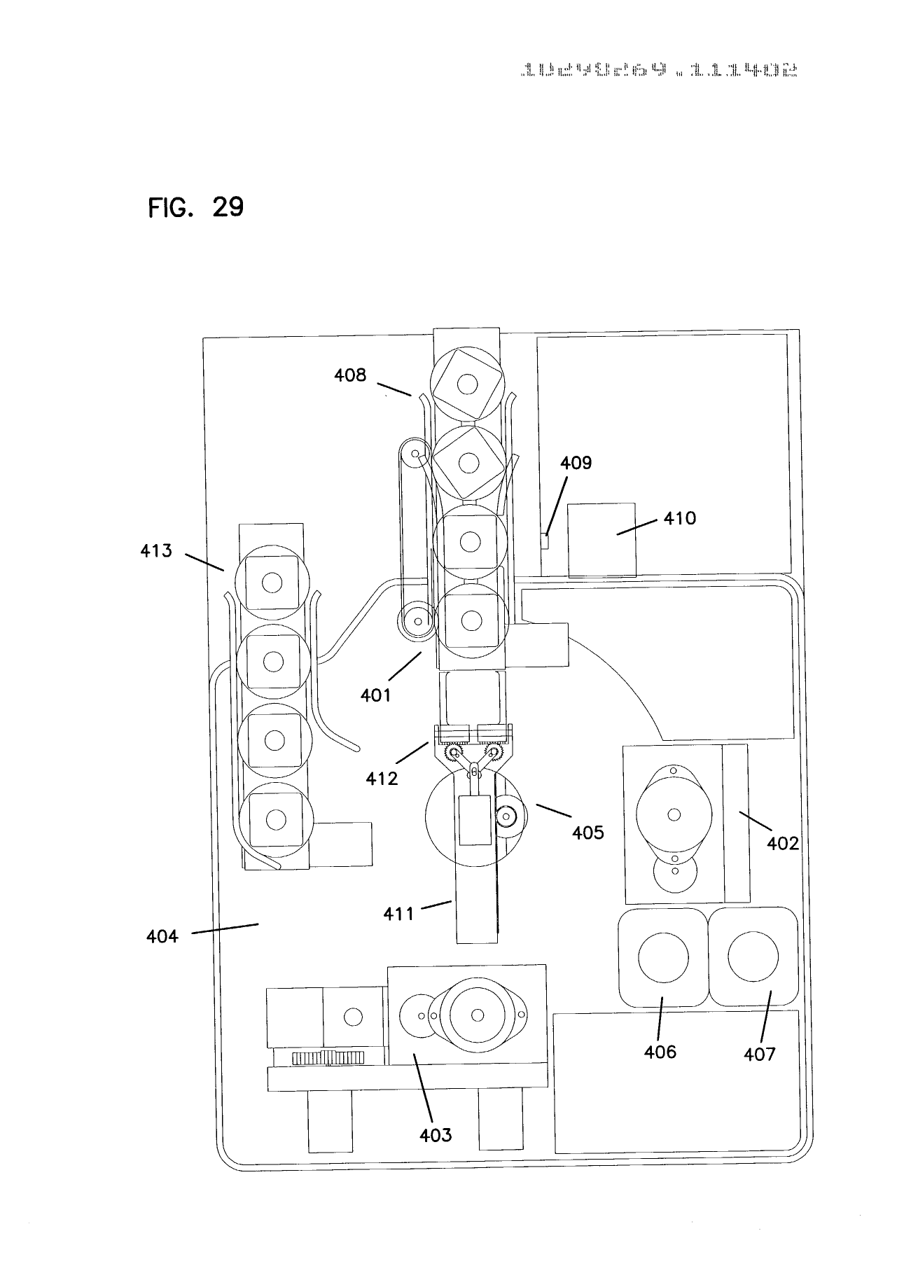

FIG. 29 shows an embodiment in which a cellular sample is delivered to the instrument contained within the sample collection device 100. A cell fixative solution within the sample collection device preserves the cells and preconditions them for staining. Upon transfer from the loading station 401 to the processing station 402 of the instrument, the fixative can be drained from the cell collection device 100 into a waste container 407 and the residual fixative is removed by exposing the cells on the supporting surface of the cell collection device 100 to a wash solution that is provided from an external bulk container 406.

The labeling reagents described herein can then applied to the cells from a compartment 107 located within the closure element 104 of the sample collection device 100. The cell collection element 101 with adhering stained cells can then be transferred to a reading station 403 where the locations and fluorescent intensities of any fluorescent objects on the surface of the cell collection element 101 can be determined in a spatially resolved manner.

As shown in FIG. 29, samples are introduced into the instrument via a loading station 401, which provides a point where multiple samples can be queued for entry into the instrument and serves as a barrier that isolates the processing section of the instrument from external influences.

The queuing function of the loading station serves two primary purposes. One of these purposes is that it decouples operator intervention from the timing cycle of the sample processing, as a queue allows new samples to be introduced at any time subject only to the limitation of queue capacity.

The second purpose of the queuing function is to precondition the samples before they enter the processing section of the instrument. Environmental parameters such as temperature can have a substantial effect upon the time required to perform the staining, the degree of staining obtained and, in some cases, the ability of the stain to discriminate between various cellular constituents. To ensure consistency in the processing of samples, and thus to ensure that the results obtained from the processed samples can be compared on a common basis, those environmental parameters that affect the processing results must be controlled within suitable limits.

Similarly, the loading station 401 serves to isolate or buffer the processing section 404 from the external environment because the processing section can be completely enclosed except for a small port through which samples are transferred. A door, hatch or load lock mechanism of known design can be used to close the transfer port when samples are not being transferred should this added degree of isolation be preferred in a particular application. The loading station 401 eliminates the need for a user to directly access the processing section 404 and therefore eliminates or reduces the environmental transients associated with such direct access.

A transfer mechanism 405 of known design is employed to move the sample between stations in the instrument. In the embodiment being described, the same transfer mechanism is employed to move the sample between the various stations. However, the functions of the processing and reading stations can be integrated into a single station, thus obviating the need for a means of transferring a sample between them. The preferred embodiment for a specific instance of the invention depends upon the particular application environment in which the invention is to be used.

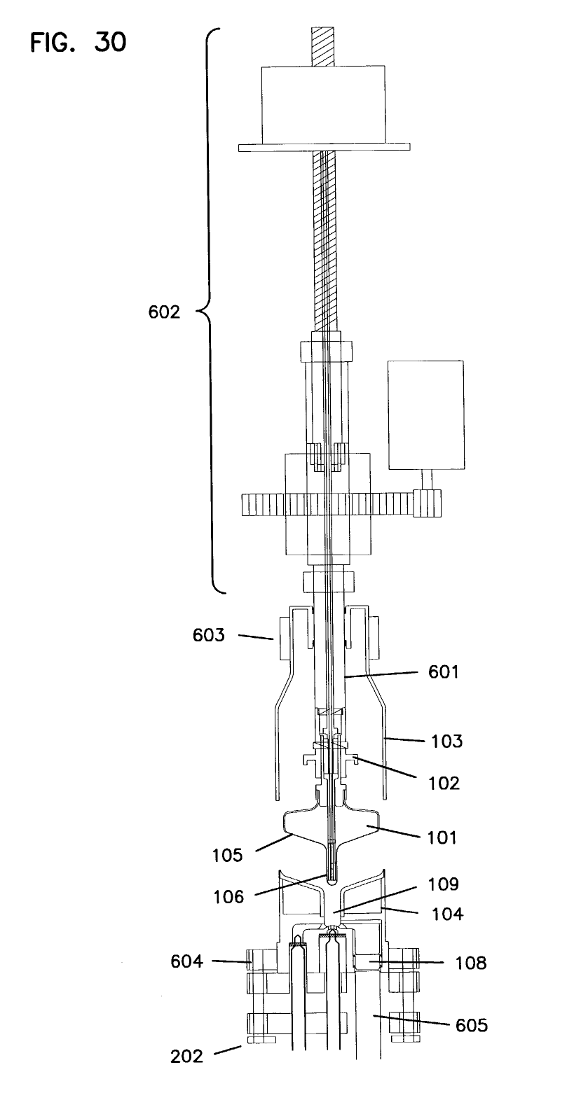

A suitable processing station for this configuration is illustrated in FIG. 30. The transfer mechanism 405 delivers the sample collection device to the processing station 402 in an orientation such that three access ports 108, 109 and 110 in the end closure 104 and the coupling element 102 on the opposite face of the sample collection device are aligned with the corresponding features in the processing station. Additional features in the processing station mate with ridges and flats (not shown) incorporated into the outer surface of the sample collection device 100 in a manner that allows clamp mechanisms 603 and 604 to secure the container 103 and end closure 104, respectively, to the processing station 402.

When the collection device 100 is properly positioned and is secured in the processing station 402 by clamp 604, a shaft 601 is extended from the processing station 402 and engages the coupling element 102 on the collection device. Rotating 601 disengages the sampling element 101 from the shell 103 of the collection device 100 and separates the shell 103 from the end closure 104. Slightly retracting shaft 601 lifts the shell 103 from the end closure 104 to a position where the shell 103 is secured in place by clamping mechanism 303. This leaves the sampling element 101 suspended above the end closure 104, the face 105 of which is contoured in a manner that mirrors the contour of the face of the sampling element 101. The contoured face of the end closure 104 forms a well into which reagents can be introduced and into which the sampling element can be dipped for the purpose of performing the staining and washing reactions.

At this point, the reaction well contains the fixative solution that was applied to the sample prior to introduction of the sample into the instrument. The fixative is drained and the well is filled with wash solution and the sampling element 101 is lowered until the face of the element is immersed in the wash solution. Washing can be facilitated by rotating or oscillating the sampling element longitudinally or, alternatively, by introducing a pulsating stream of air into the sampling element via a channel in the coupling element. Upon completion of the washing, the sampling element is raised above the well and the used wash solution is drained as before. The wash cycle may be repeated as needed until the excess fixative has been removed from the sampling element.

Upon removing the fixative solution, the staining reagent is introduced into the reaction well. In the configuration illustrated, the reagent is contained in a break-seal pouch 107 within the end closure 104 of the sampling device 100. As suggested in FIG. 30, the instrument extends a plunger 605 through an access port 108 in the end closure 104 of the sampling device 100 to compress the reagent pouch 107. This pressure causes the break-seal to rupture, thus discharging the contents of the pouch into the reaction well. The staining reaction is carried out by immersing the sampling element into the pool of reagent in the reaction well and mixing as described above. At the conclusion of the staining reaction, the sampling element is elevated above the reaction chamber, the spent reagent is drained from the chamber, and the sampling element washed as described above. The washed sampling element can be dried in a stream of warm, dry air. Other dispensing means such as spraying, aspirating, nebulizing or pipetting may be employed to deliver fixative, staining reagents and wash solution to the sampling element 101. Such alternative dispensing means will require that the design and operation of the process station 402 will differ from that described above.

The rate of drying of the sampling element is determined by the amount of residual fluid on the surface of the sampling element and by the volatility of this fluid. The drying rate can be increased by rinsing the sampling element in a water miscible, volatile cell fixative solution such as ethanol or isopropanol prior to drying.

Upon completion of the staining, washing and drying, the sample container is re-closed, the sampling device 100 is disconnected from the vertical shaft 601, and the device 100 is moved to the reading station 403 by the transfer mechanism 405.

A function of the reading station 403 is to measure the fluorescence at each point on the surface of the face of the sampling element 101. To this end, either of two optical systems are used depending upon whether, in the particular application, it is necessary or desirable for the operator to be able to visually view the surface of the sampling element. If, as is the case in research and certain other specialized applications, it is desirable for the operator to be able to visually view the surface of the sampling element, the optical system consists of a video camera or viewing tube coupled to an appropriate microscope objective lens.

An optical system based upon a CCD video camera, for example, imposes less stringent demands upon accuracy, precision and resolution than does a flying spot scanner because, in the former case, the effects of several of the predominant types of positioning error can be compensated for during the processing of the acquired data. Similarly, the means (not shown) employed for determining the position and alignment of the optical system relative to the surface of sampling element 101 depends upon the type of optical system employed.

Processing a Cell Suspension

Cell suspensions can be processed manually, as described for example in U.S. Ser. No. 09/981,185, which is expressly incorporated by reference herein. A triage device can be configured to capture cells found in a cell suspension. A triage device can include a sample well configured to hold the cell suspension, a membrane filter that is a thin plastic filter having pores that are sized to accommodate the cell suspension, a fluid flow control layer that limits fluid velocity, and a bulk absorbing layer. The cells can be optically analyzed using the reagents described herein while the cells remain on a surface of the membrane filter.

The triage device described herein is intended to provide a simple, rapid means of determining whether a cellular sample should be referred for further, more detailed cytological evaluation. A membrane filter can be positioned over a flow control layer that limits fluid velocity to ensure more complete wetting of the membrane filter. A bulk fluid absorber layer can be positioned under the flow control layer to capture most or all of the fluids that pass through the upper layers of the triage device. A sample well can be provided atop the membrane filter.

In use, a volume of cell suspension is transferred into the sample well, where the cells are allowed to settle across the membrane. Flow through the membrane will increase as the membrane becomes fully wetted. Once the membrane has been fully wetted, fluid flow through the membrane will remain essentially constant. Once the cell suspension has been processed, subsequent reagents can be applied as described herein.

An optical image of the sample can be captured and can be examined pixel by pixel to identify possibly abnormal cells and their relative location within the cervix. Alternatively, once the membrane has been loaded with cellular material, it can also be examined optically by using, for example, a video microscope to visually inspect the sample.

Cell suspensions can also be processed mechanically. Suitable mechanical processing is described for example in U.S. Pat. Nos. 5,143,627 and 5,240,606, each of which are explicitly incorporated by reference herein.

Preparing Slides

Cellular samples such as cervical cells can be obtained from a cell suspension and then transferred to a microscope slide for analysis. The cells can be retrieved from the cell suspension using an inexpensive, easy to use device that requires no instruments or ancillary devices, minimal operator skill and training, and is potentially sufficiently low cost that it is suitable for use in mass screening programs. Cells can be transferred using devices that employ gravity settling or by devices that employ pressurization. See for example U.S. patent application Ser. No. 10/004,704, which is expressly incorporated by reference herein.

The device illustrated in FIG. 25 includes a plunger 518 that moves slideably within a compartment 512 which in turn communicates with a second compartment 522 by way of a slot or hole 524. The compartment 512 further incorporates a recess 520 in one wall that communicates with the slot or hole 524, but which does not extend to the bottom of the chamber 512 that is formed by the microscope slide 536.

The cell suspension from which the microscope slide 536 is to be prepared may be prepared separately and transferred into the chamber 512, or it may be prepared in-situ by agitation of a cell collection device in preservative fluid previously placed in the chamber 512.

When the plunger 518 is initially inserted into the chamber 512, features schematically illustrated as o-rings 525, 526 form a liquid tight seal between the plunger 518 and the chamber 512 except in the area of the recess 520. Advancing the plunger 518 into the chamber 512 causes any trapped air to be vented to atmosphere through the recess 520 until the plunger 512 contacts the surface of the cell suspension. Further advancing the plunger 512 causes cell suspension to be displaced from the chamber 512, through the recess 520 and the slot 524 into the chamber 522 until the seal 526 reaches the bottom of the recess 520. When the seal 526 reaches the bottom of the recess 520, it forms a closed compartment 516 containing a volume of cell suspension. The volume of cell suspension contained in the compartment 516 are determined by the dimensions of the compartment which are in turn determined by considerations previously described.

The plunger 518 can include a body 519 that fits into the chamber 512 and forms a fluid tight seal with the walls of the chamber 512 by means of the sealing features 525, 526. The bottom end of the plunger 518 can include a membrane filter 528 such as a track etch filter having a 5 to 10 micron pore size. The membrane filter 528 can be backed by a relatively thin absorbent pad 530 which, in turn, can be supported by a porous frit or similar structure 532.

The plunger 518 can also include an absorbent material 534 that will absorb a portion of the diluent present in the cell suspension sample and thus help pull cells onto the membrane filter 528. The absorbent material 534 can be formed from any suitable blotter or absorbent material. The desired sub-sample volume of cell suspension is trapped and pressurized between the face of the plunger 518 and the chamber walls.

When the seal 526 reaches the bottom of the slot 524, the closed compartment 516 containing a predetermined volume of cell suspension is formed. Continued advancement of the plunger 518 into the compartment 516 generates hydraulic pressure that forces the trapped fluid through the filter 528 and into the absorbers 530 and 534. This fluid motion is further enhanced by the absorbent action of the absorbers. During this process, the cells trapped in the chamber 516 are captured on the surface of the filter 528. Absorption of fluid causes the absorbent pad 530 to swell thus imparting a slight dome shaped distortion to the filter 528.

At the bottom of the plunger stroke, the membrane 528 makes contact with the microscope slide 536. The fluid swollen pad 530 acts as a compliant element that ensures that the membrane 528 makes good contact with the slide 536 as the plunger 518 bottoms out in its stroke, much in the manner of tampo or pad printing. The microscope slide 536 can be selected such that the trapped cells adhere to it better than they do to the membrane 528, so that the captured cells are transferred to the slide 536 and remain as a defined and nearly dry spot on the slide 536 when the slide 536 is separated from the cell transfer device 510. The resulting slide 536 can then be fixed, stained and evaluated as described herein.

### EXAMPLES

### Example 1

This example demonstrates markers suitable for detecting dysplastic cervical cells. In particular, two cell surface receptors, transferrin receptor (TfR) and epidermal growth factor receptor (EGFR) were tested for their efficacy in identifying abnormal cells.

Materials and Methods

Cervicovaginal specimens in Autocyte liquid preservative medium were obtained from patients under IRB approval at several clinical sites. Specimens were diagnosed using cytological criteria, as outlined below:

| Classification | Criteria |
|---|---|
| normal | within normal limits (WNL) |
| atypical | atypical cells of undetermined significance (ASCUS) |
| abnormal | low grade squamous intraepithelial lesions (LSIL) |
| abnormal | high grade squamous intraepithelial lesions (HSIL) |

Monolayer preparations of cervical epithelial cells were prepared using a cytospin (Shandon Cytospin 3, Shandon, Inc, Pittsburgh, Pa.). An aliquot of the resuspended specimen was added to a small well cytospin funnel and cells were distributed onto positively charged glass slides (Super Frost Plus, Fisher) using centrifugal force (1000 rpm, 2 min, 25° C.). Immediately following cytospin preparation, cells were spray fixed with Shandon Cell fix, followed by several dips in ethanol.

For EGFR staining, cells were permeabilized with a detergent and slides were then blocked with a non-serum containing blocking reagent. Anti-EGFR antibody conjugated to a fluorophore was added and slides were incubated in the dark at 25° C. Slides were then washed in PBS, air-dried, fixed by spraying with CytoPrep fixative (Fisher), and coverslipped for the capture of images of the immunofluorescently stained cell sample. Immunostained cervical epithelial cell monolayers were scanned and fluorescent images were captured automatically, with the AcCell® automated microscope, equipped with Samba Technologies image analysis software.

For TfR staining, cytospin-prepared slides containing cell monolayers were immersed in a Tris-buffered saline solution and blocked with a goat serum solution. Cells were incubated with anti-TfR antibody conjugated to a fluorophore for a defined period of time, washed with TBST. Cells were washed, mounted in Fluorosave and coverslipped for image capture.

Fluorescent images were captured at 10×magnification using the AcCell® microscope and Spot digital camera, under control of Samba image analysis software. The locations on the slide from which fields of view were captured were automatically recorded by the AcCell system. Fluorescently stained cells were quantified and reported by the software. After image capture was completed, coverslips on immunostained slides were removed by immersion in water.

Slides were counterstained using a standard Papanicolaou staining method. A cytotechnologist, using cytological criteria, then screened the Pap-stained slide. A cell-by-cell review based upon morphologic criteria of fluorescently immunostained cells was also conducted, using the locational guidance feature of the image software. Sensitivity and specificity values were assigned to patient specimens, based upon immunofluorescent staining in combination with cytological review.

Results for EGFR

Results from immunostaining of 63 patient specimens for detecting EGFR are shown in Table 1. A positive result was documented in those cases with 1 or more immunostained cells. This study illustrates the efficacy of EGFR for the detection of high-grade lesions. All 12 cases diagnosed HSIL or CIN 2/3 (100%) were positive for EGFR staining. Specificity in this study was 73.3% based on the use of a single antibody and simple, non-algorithmic software detection of fluorescence over an experimentally validated fluorescent intensity.

TABLE 1
EGFR Immunostaining of 63 Cervical Cytology Specimens

| Diagnosis¹ | #Cases | #EGFR-Positive | % EGFR Positive |
|---|---|---|---|
| HSIL² | 12 | 12 | 100 |
| LSIL | 27 | 23 | 85.2 |
| ASCUS | 9 | 7 | 77.8 |
| WNL | 15 | 4 | 26.7 |

¹All diagnoses were based upon cytological criteria
²Includes 5 cases with a histological diagnosis of CIN 2/CIN 3 or CIS

| | |
|---|---|
| Sensitivity, HSIL: | 100% |
| Specificity: | 73.3% |

Results for Transferrin Receptor

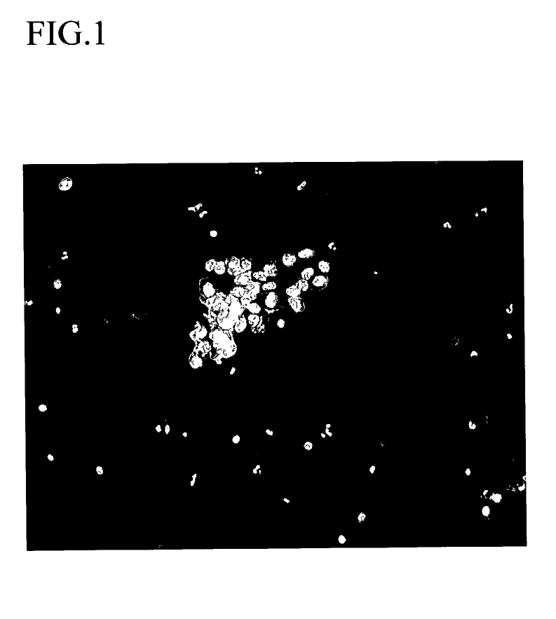

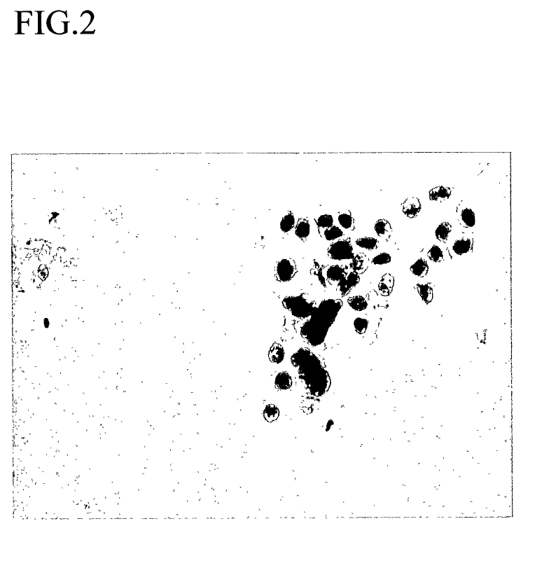

138 patient samples representing four cytologic categories defined by Bethesda system criteria were immunostained. A sample with 1 or more fluorescently reported objects constituted a ‘Positive’ result. In this study, probing for TfR detected all 3 cases of squamous cell carcinoma, as well as 15 of 16 cases diagnosed HSIL. Assay sensitivity for cases with a diagnosis of HSIL or higher was 94.7%. FIG. 1 represents a transferrin immunostaining of a group of abnormal cells that was diagnosed as HSIL. FIG. 2 shows the results of Papinicolaou staining the same group of abnormal cells, after the immunostaining illustrated in FIG. 1.

Discussion

Both the EGFR data set and the TfR study were based on software algorithms that were functionally designed to detect fluorescence of a particular wavelength (e.g., color), over a quantified, experimentally derived intensity level. In reviewing all “False Positive” (FP) cases that contributed to sub-optimal specificity, objects fluorescing over threshold fell into 1 of 6 categories. These categories included reactive squamous intermediate cells (“Reactive SI's”), some dying squamous or endocervical cells, a few metaplastic cells, polymorphonuclear cells (PMNs), and cellular artifacts and debris.

Alterations of protein expression in cervical epithelium occurring as a consequence of HPV-induced cellular transformation are also well recognized. One protein known to be up-regulated as the result of HPV E5 gene expression is EGFR. In one of the two studies reviewed here, EGFR demonstrated a 100% sensitivity for the detection of HSIL in a fluorescent assay.

In another study, TfR showed similar efficacy in detecting high-grade cervical cancer precursors, with a sensitivity of 94.7% for cases with a diagnosis of HSIL, CIS or invasive carcinoma. This study shows that transferrin receptor, like EGFR, provides an effective biomarker for the early detection of cervical cancer precursor lesions.

Because it is likely that these two receptor proteins represent discrete and independent protein expression events, these studies demonstrate that an assay based upon the combination of EGFR and TfR can have increased sensitivity and/or specificity, compared with assays based upon either biomarker alone.

### Example 2

This Example demonstrates the feasibility of developing an integrated system for identifying abnormal cells in cervical cell samples, which allows cervical cancer screening. An automated fluorescent slide imager and analyzer were developed using the Accell workstation (described above) driven by an image analysis software program (Samba Technologies, France). The software and image archiving was driven by a standard personal computer.

The cell-by-cell sensitivity of both epidermal growth factor receptor and transferrin receptor was high enough to demonstrate their usefulness as markers for cervical dysplasia. Application of these markers individually to a slide posed several problems. Both of the proteins labeled by these antibodies are expressed to varying degrees in normal endocervical cells.

Materials and Methods

Patient samples were collected prior to colposcopy exams in Cytorich liquid-based solution (TriPath Imaging) and stored at 4° C. for no more than 3 weeks prior to staining. These samples represented women with previously assessed cervical cell abnormalities who were present to visualize their cervix under standard colposcopic conditions. Samples were collected prior to the application of acetic acid to the cervix.

After gentle agitation of the liquid vial containing cervical cells, 100 μl was removed and placed into a prepared Shandon cytofunnel. Samples were spun at 1000 rpm at room temperature for 3 minutes utilizing positively charged glass slides. Slides were immediately removed and sprayed once with Shandon CytoFix cell fixative and allowed to air dry for 4 minutes. Slides were then dipped twice in 95% ethanol and twice in 70% ethanol to remove any polyethyleneglycol and placed in a wash buffer for 1 minute to remove residual alcohol.

Slides were gently tapped off and 20 μl of primarily conjugated antibody against EGFR (Pharmingen) was applied for 15 minutes in the dark. Slides were rinsed with distilled water and placed in wash buffer on a rocker for 2 minutes to remove non-specifically bound antibody. Buffer was tapped off and slides were mounted using Gelmount (Biomeda) and a standard glass coverslip. After fluorescent imaging, coverslips were removed by gentle agitation in warm distilled water and pap stained with a standard protocol utilizing modified Gill's heamotoxylin.

Imaging

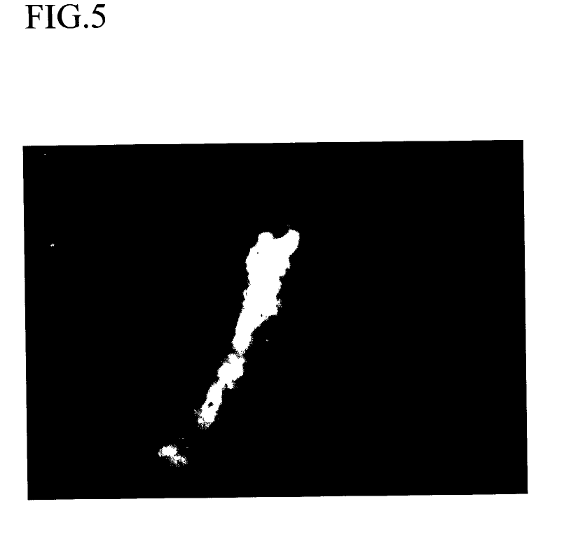

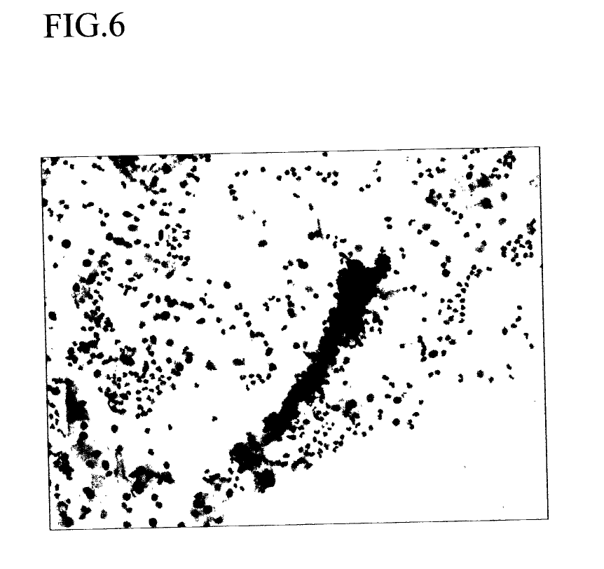

Slides were imaged utilizing a modified Olympus BX-40 transmitted light microscope (Accell workstation) with fluorescent attachment. The excitation light source was a standard mercury halogen bulb. A general wide-band phycoerythrin filter cube was initially utilized and images were captured with a Spot Advance digital camera (Research Diagnostics). FIGS. 5 and 6 illustrate cells from cervical cytology specimens stained with anti-EGFR and subsequently PAP stained, respectively.

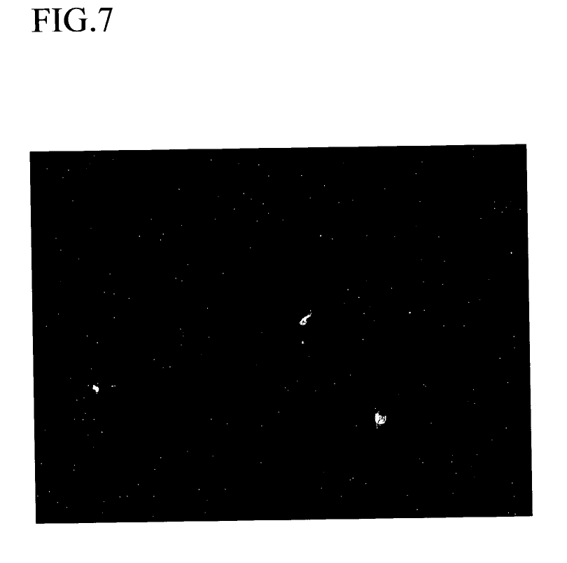

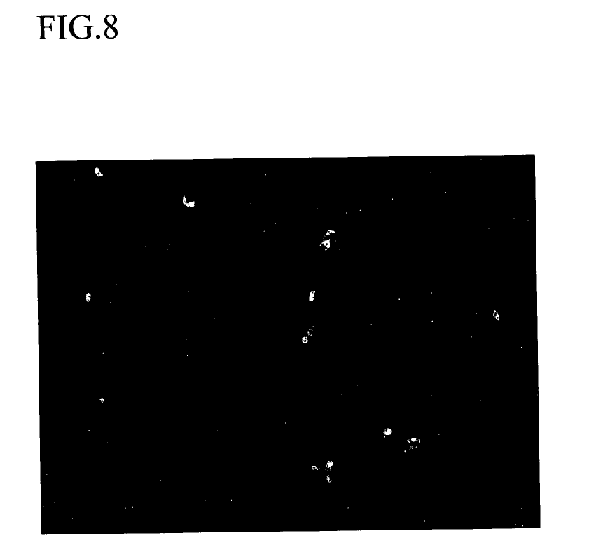

Using a protocol identical to the one for epidermal growth factor receptor, monoclonal antibodies against transferrin receptor yielded excellent results on a cell-by cell basis. Imaged dysplastic cells showed an approximate 3× increase in transferrin receptor protein expression. This expression was quantified through mean fluorescence analysis versus normal squamous intermediate cells imaged in the same fields. The mean fluorescence for normal squamous-intermediate cells was 28.3 on a scale of zero to 255. FIGS. 7 and 8 illustrate both normal and neoplastic cells imaged using the same method.

### Example 3

A variety of possible cervical cell markers were assessed to develop a correlation of a given marker to cervical dysplasia. This assessment employed a novel method that enabled cell-by-cell correlation with cervical cytology. Employing an automated cytology workstation (AcCell, Accumed Intl., Chicago, Ill.) patient samples were stained with a given marker and visualized under a 10× objective. Fields of view of interest were imaged and stored with a high resolution digital camera (Spot, Research Instruments) and the exact coordinates of the field were archived utilizing the AcCell workstation. This enabled future re-screening of the fields after subsequent pap staining.

List of Immunochemical Markers Assessed

| | |
|---|---|
| Insulin-like Growth Factor - 1R | Heat Shock Protein 70 |
| GAPDH | Ki67 |
| Epidermal Growth Factor Receptor | p16 |
| Cyclin D | erbB2 |
| Cyclin B1 | MN |
| Cdc6 | Cdc2 |
| McM5 | Cytokeratin 13 |
| Cytokeratin 19 | Transferrin Receptor |
| Proliferating Cell Nuclear Antigen | NMP 179 |

Patient Sample Screening

Sample Retrieval and Slide Preparation Methods

Patient samples were collected from both the Women's Health Center (University Hospitals of Cleveland) and surgical suites associated with McDonald Women's Hospital, University Hospitals of Cleveland. Patients who signed institutional review board approved consents presented to the Women's Health Center for a colposcopic examination of their cervices due to a recent abnormal pap screen during annual exams. It is standard care at University Hospitals of Cleveland Women's Health Center not to perform a second cervical cell collection prior to colposcopy. This aided in retrieving newly exfoliative tissue with a high probability of cervical cell abnormality.

Cells were immediately placed into a liquid-based fixative/preservative (TriPath Imaging) and cell concentrations were standardized to 100,000-200,000 cells/ml. Samples were stored at 4° C. for a maximum of 4 weeks for future molecular assessment.

Slide Preparation

Slide preparations were performed with a Cytospin 3 (Shandon Scientific) by placing 100 ul of cell suspension in a clean, reusable funnel and spinning at 1000 rpm for 3 min at room temperature. Positively charged glass slides (Fischer Scientific) were utilized at all times. Cells were fixed a second time utilizing a non-aerosol fixative (Cell-Fix, Shandon Scientific) immediately upon completion of the cell spin. Slides were then allowed to air-dry for a maximum of 5 minutes at room temperature. This protocol allowed greatest cell retention on the glass slides during the cover-slip removal procedure. Prior to application of any antibody, slides were dipped twice in 95% ethanol and twice in 70% ethanol to remove any polyethylene glycol from the slide surface.

Application of Immuno-Markers

It was determined during the marker screen that two markers tightly linked to cervical cell growth and proliferation would yield the highest probability of optimizing a functional assay for cervical cell abnormality. The ligands for these markers, epidermal growth factor receptor and transferrin receptor have been reported to be necessary for proper growth of cervical cells in culture. The antibodies used were both primary conjugates with phycoerythrin ( Pharmingen, San Diego, Calif.), a large fluorophore with appropriate quantum efficiency. Antibodies were applied “neat”, with 20 ul/6 mm cytospin “pellet”.

Results

Marker Selection

TABLE 2
Results For Individual Markers.

| | Minimum | | |
|---|---|---|---|
| Marker | Amplification | Localization | Signal Detection Level |
| IGF-1R | Tertiary | NA | None Detected |
| Heat Shock | Binary | Cytoplasmic | Inconsistent |
| Protein 70 | | | |
| GAPDH | Binary | Cyto/Nuclear | Good. Positive in Normal |
| | | | and Abnormal Cells |
| Ki67 | Tertiary | NA | None Detected |
| EGFR | None | Membranous | Good. |
| p16INK4A | Binary w/ | Cyto/Nuclear | Low and inconsistent. High |
| | Enzymatic | | Background. |
| Cyclin D | Tertiary | NA | None Detected |
| erbB2 | Tertiary | NA | High non-Specific Binding |
| Cyclin B1 | Tertiary | NA | None Detected |
| MN | Binary w/ | NA | None Detected |
| | Enzymatic | | |
| Cdc6 | Tertiary | NA | None Detected |
| Cdc2 | Binary w/ | Nuclear | Low and Inconsistent. High |
| | Enzymatic | | Background. |
| Mcm5 | Binary w/ | NA | None Detected |
| | Enzymatic | | |
| Cytokeratin | Binary | Cytoplasmic | Good. Positive in Normal. |
| 13 | | | |
| Cytokeratin | Binary | NA | None Detected |
| 19 | | | |
| Transferrin | None | Membranous | Good. |
| Receptor | | | |
| PCNA | Binary | Nuclear | Inconsistent |

Table 2 shows the results for individual markers tested for their correlation to cervical neoplasia. The reported results are based upon the use of immunofluorescence techniques for detection. Levels of amplification are based upon the use of a primary IgG raised against the antigen of interest (usually murine derived) and then an anti-murine secondary (usually goat or rabbit derived w/ or w/o phycoerythrin) and, optionally, a subsequent anti-goat-phycoerythrin tertiary.

These results demonstrate that epidermal growth factor receptor is minimally expressed in fully differentiated squamous cells and intermediate squamous cells but is expressed in normal endocervical cells to a high degree. These results demonstrate that epidermal growth factor receptor (EGFR), transferrin receptor (TfR), glyceraldehyde-3-phosphate dehydrogenase (GAPDH) are suitable markers for proliferating cells in a sample of cervical cells.

Patent Samples

TABLE 3
Results From Analysis of Patient Samples

| | Slide | Assay | |
|---|---|---|---|
| Patient # | Diagnosis | Result | Objects I.D. on WNL Slides |
| 1 | SIL | + | N.A. |
| 2 | Reactive | − | N.A. |
| 3 | WNL | + | Debris w/PMN |
| 4 | WNL | − | N.A. |
| 5 | Reactive | − | N.A. |
| 6 | WNL | − | N.A. |
| 7 | WNL | − | N.A. |
| 8 | HSIL | + | N.A. |
| 9 | HSIL | + | N.A. |
| 10 | WNL | − | N.A. |
| 11 | Atypical | + | N.A. |
| 12 | WNL | + | Overlapped Squamous Cells |
| 13 | WNL | + | Debris w/PMN |
| 14 | WNL | − | N.A. |
| 15 | WNL | − | N.A. |
| 16 | WNL | − | N.A. |
| 17 | LSIL | − | N.A. |
| 18 | WNL | − | N.A. |
| 19 | WNL | − | N.A. |
| 20 | WNL | − | N.A. |
| 21 | WNL | + | Debris w/PMN |
| 22 | HSIL | + | N.A. |
| 23 | SIL | + | N.A. |
| 24 | Atypical | + | N.A. |

In this table, results for false-positives (i.e. WNL slides w/ objects meeting fluorescent criteria) are shown on right hand column.

Discussion

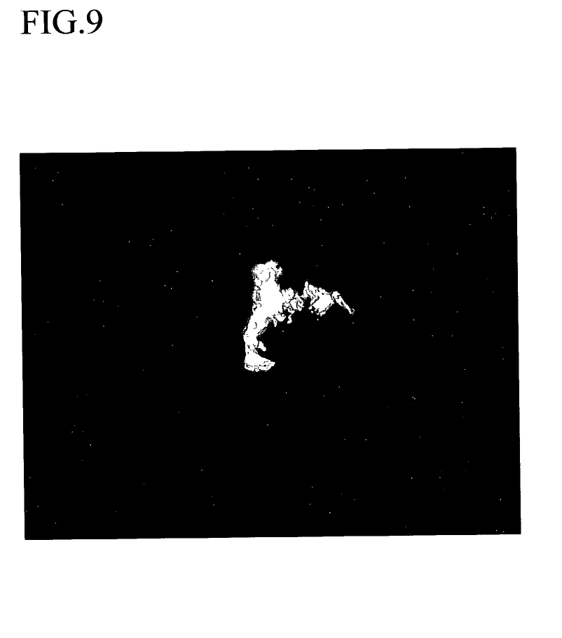

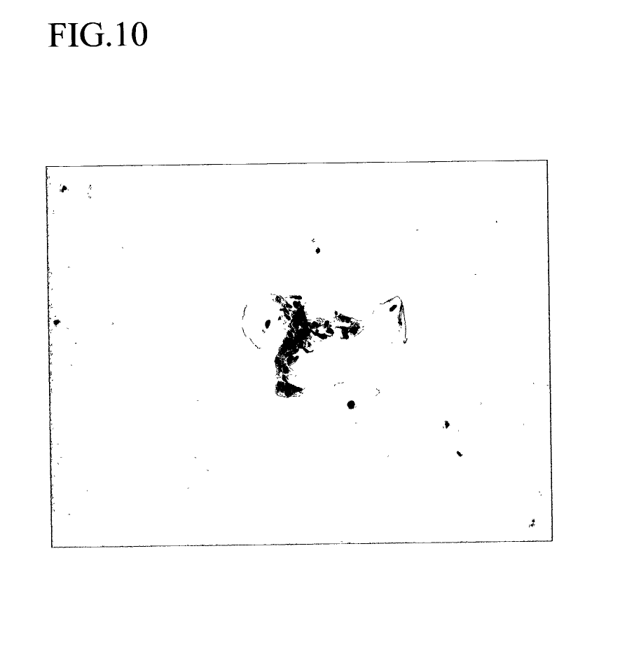

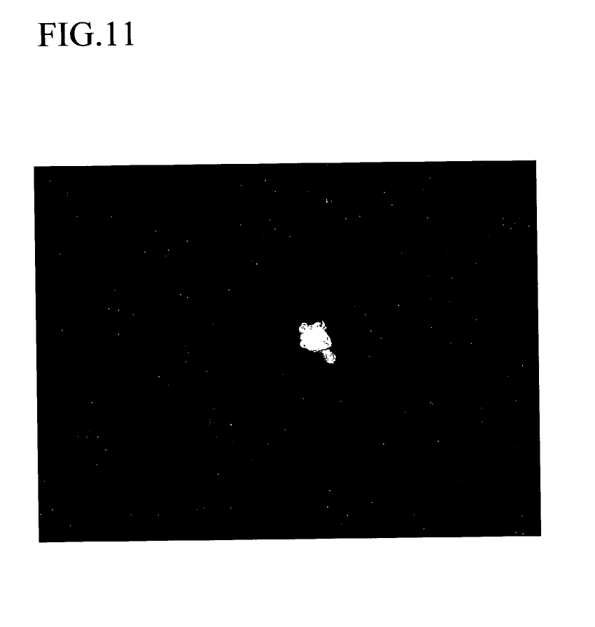

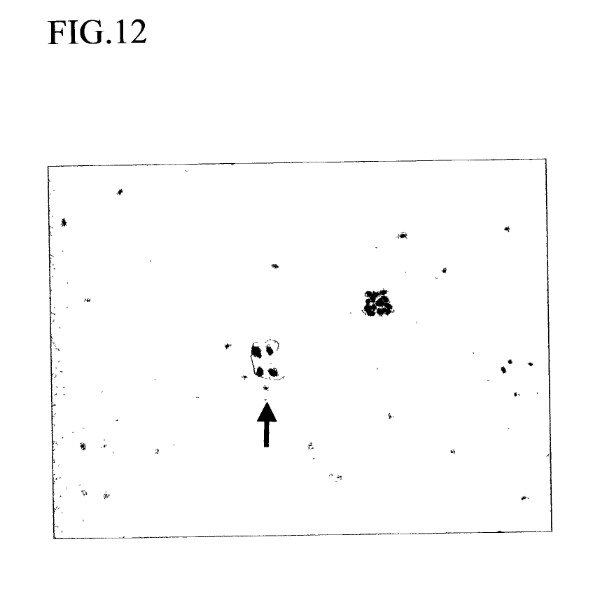

Either or both of epidermal growth factor receptor or transferrin receptor can be employed as positive markers for cervical neoplasia if the method also employs a marker that is both sensitive and specific to endocervical cells. This marker for endocervical cells was found to be an antigen expressed only in glandular cells of squamous origin, cytokeratin 7/8 (which can be identified by an antibody called Cam5.2 (Pharmingen)). The sensitivity and specificity for this antibody in cervical cytology specimens is near 100% and its ease of use makes this an attractive “subtractive” marker in the present method. FIGS. 9, 10, 11 and 12 show images of endocervical cells labeled with Cam5.2. Corresponding pap images are to the right.

A second characteristic often encountered on cervical cytology is the presence of debris and hyperkeratotic cells, which are hyperdifferentiated, anucleated, normal squamous cells. Both of these entities exhibit apparent non-specific binding of murine antibodies, which leads to false positive results when quantifying immunofluorescence. To alleviate the problem associated with these objects the addition of a third marker and combined image analysis software was used to remove them from the analysis. This was accomplished by addition of the DNA minor groove binding dye 4,6-diamido-2-phenylindole, dihydrochloride (DAPI), which predominately binds to intact, double stranded DNA.

Image analysis software was employed to first identify blue objects, which represent intact nuclei. Analysis was then performed to associate any green fluorescence due to the labeled endocervical cells with these nuclei and subtract these cells from analysis. The next step quantified red pixels in a halo around the blue nuclei. This identified cells labeled by the phycoerythrin (i.e. the positive marker) on the anti-EGFR antibody for which fluorescence intensities were above a given threshold.

The combination of the markers employed in this embodiment of the present assay together with the hardware/software provided a unique opportunity to automate the process of slide analysis. Utilizing the Samba software to image 100% of a prepared slide labeled according to the present method, resulted in full slide imaging and archiving in approximately 6 minutes. Combined with the slide preparation protocol, which takes approximately 3 minutes, and the assay, which takes approximately 16 minutes, the present method provides a protocol for “real-time” analysis of cervical samples. In a clinical environment this approach could be used as a point-of-service screening test for cervical pre-cancer.

Utilizing this 3 color, multi-parameter approach 12 slides were imaged. With a “zero-sum” threshold (i.e. one positive object signifies a positive slide) a slide based sensitivity of 88% was seen when taking into account all grades of abnormality. Stratification of abnormality gave a 66% sensitivity (2/3) for low-grade disease and a 100% sensitivity level for high grade disease (3/3). Conversely, a slide-based specificity of 75% was achieved. If a “worse case” slide assessment was used as the correlation parameter a sensitivity of 83% and specificity of 83 % was seen. This method of correlation permitted rare abnormal events present of either histology or other pap slides from the same patient vial to represent true disease of the cervix. Table 3, above, shows the assay results for these cases.

### Example 4

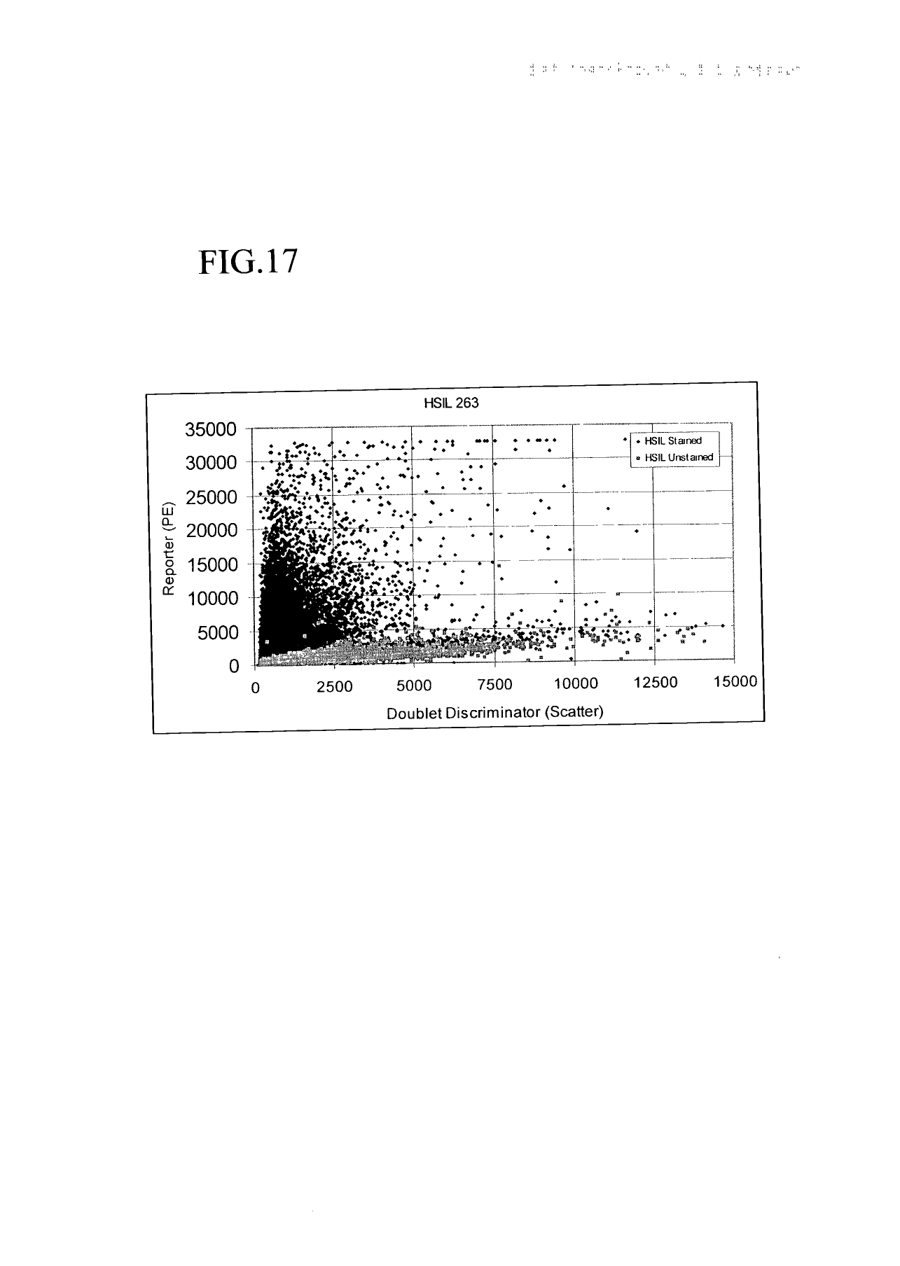

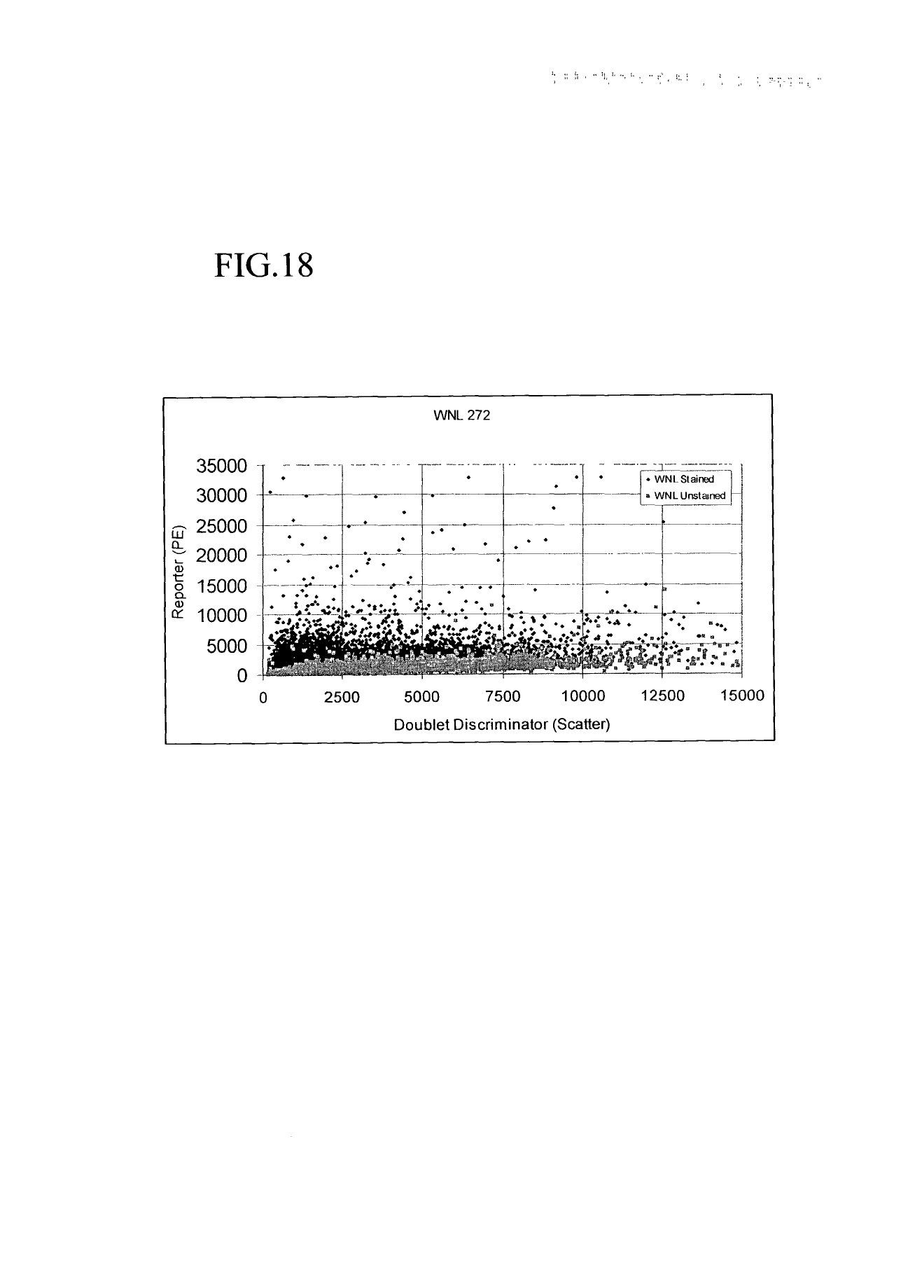

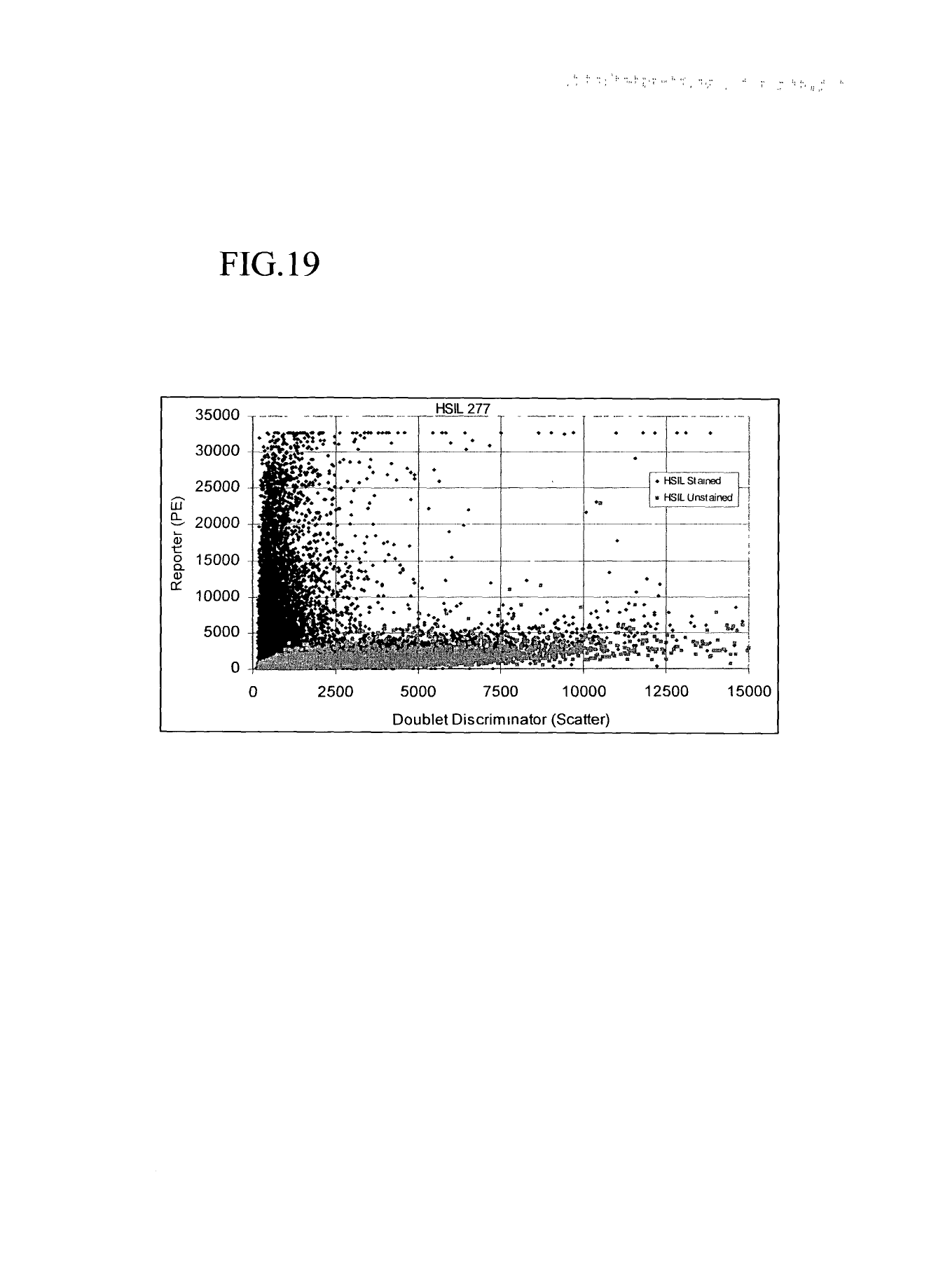

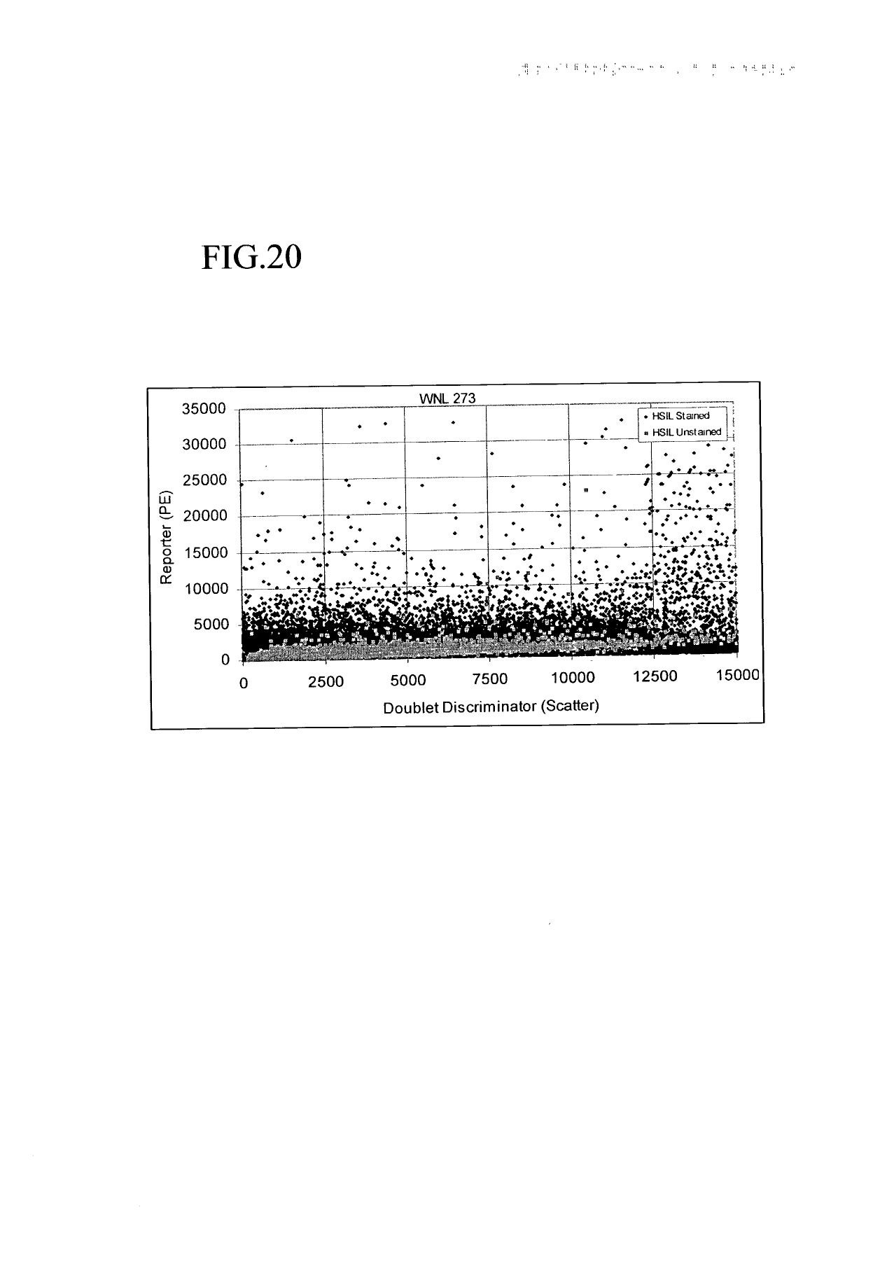

Samples of abnormal (HSIL) cells and normal (WNL) cells were analyzed using flow cytometry. In particular, the cells were suspension stained with anti-TfR labeled with phycoerythrin and were washed once using PBS in a sedimentation process. The cells were then run through a flow cytometer (Luminex 100) and the resultant fluorescence was captured and subsequently plotted. FIGS. 17, 18, 19 and 20 represent two samples deemed to be abnormal and two samples deemed to be normal, respectively.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

While the invention has been described with reference to specific embodiments, it will be apparent to those skilled in the art that many alternatives, modifications and variations may be made. Accordingly, the present invention is intended to embrace all such alternatives, modifications and variations that may fall within the spirit and scope of the appended claims.

We claim:

1. A method for identifying abnormal cells in a cytological sample, the method comprising:
providing a cytological sample;
contacting the cytological sample with a first reagent that selectively labels proliferating cells;
contacting the cytological sample with a second reagent that selectively labels normal proliferating cells;
contacting the cytological sample with a third reagent that selectively labels cellular material; and
identifying as abnormal any portions of the cytological sample that have been labeled by the first reagent and the third reagent but not the second reagent.

2. The method of claim 1, wherein the cytological sample is contacted with the first reagent, the second reagent and the third reagent substantially simultaneously.

3. The method of claim 1, wherein the first reagent selectively labels proliferating cells by identifying a marker that is up-regulated in proliferating cells.

4. The method of claim 1, wherein the first reagent selectively labels proliferating cells by identifying a marker that is down-regulated in proliferating cells.

5. The method of claim 1, wherein the first reagent selectively labels proliferating cells by identifying a marker that is up-regulated in proliferating cells and a marker that is down-regulated in proliferating cells.

6. A method for detecting abnormal cells in a cytological specimen, the method comprising:
treating said cytological specimen with a first reagent that specifically labels a cellular constituent that differs in concentration between normal and abnormal cells;
treating said cytological specimen with a second reagent that specifically labels cell types that are not of interest, said cell types being capable of expressing the cellular constituent labeled by the first reagent;
treating said cytological specimen with a third reagent that permits discrimination between cellular and non-cellular material;
capturing an image or plurality of images of said cytological specimen after treatment of the specimen with said three reagents wherein said image or images permit discrimination between the respective areas labeled by the said three reagents;
evaluating the image(s) to localize and define objects stained with the first, second and/or third reagents; and
classifying the cells labeled with the first reagent and the third reagent, but not with the second reagent as being abnormal.

7. The method of claim 6, wherein the first reagent and the second reagent each comprise fluorescently labeled antibodies.

8. The method of claim 6, wherein the first reagent comprises a fluorescently labeled antibody directed against epidermal growth factor receptor (EGFR).

9. The method of claim 6, wherein the first reagent comprises a fluorescently labeled antibody directed against Transferrin receptor (TfR).

10. The method of claim 6, wherein the second reagent comprises a fluorescently labeled antibody directed against one or more cytokeratins.

11. The method of claim 6, wherein the third reagent comprises a fluorescent DNA stain.

12. The method of claim 6, wherein the third reagent comprises a quantitative fluorescent DNA stain.

13. The method of claim 12, wherein an intensity of fluorescent emission from the third reagent provides an estimate of the DNA content or ploidy of the cell.

14. The method of claim 13, wherein ploidy is included as a parameter in the classification of a cell as being normal or abnormal.

15. The method of claim 6, wherein the image is corrected for spectral overlap prior to image evaluation

16. The method of claim 6, wherein each reagent includes a fluorophore and where coefficients derived for each fluorophore individually and the measured values for each fluorophore in the image are used to construct a set of equations that are solved to obtain values for each fluorophore that have been corrected for the overlap between the emission spectra of the fluorophores.

17. The method of claim 16, wherein the fluorophores are selected such as to minimize the spectral overlap resulting from the fluorophores associated with the second and third reagents into the spectral emissions from the fluorophore associated with the first reagent.

18. The method of claim 6, wherein the image is processed and evaluated in a hue-saturation-luminance (HSL) color space.

19. The method of claim 6, wherein the image is segmented in the hue-saturation-luminance HSL color space in order to identify pixels to be evaluated in a red-green-blue (RGB) color space.

20. The method of claim 6, wherein the image data is evaluated using a multivariate classifier.

21. The method of claim 6, wherein individual image pixels corresponding to regions labeled by the first and second reagents are associated with the objects defined and localized by labeling with the third reagent.

22. The method of claim 6, wherein the image is analyzed on the basis of the contributions of the fluorescent emissions of all three reagents to the signal measured at each individual pixel location.

23. The method of claim 6, wherein the image is analyzed on the basis of the average contributions of the fluorescent emissions of each of the three reagents to multi-pixel regions of the image and the spatial relationships between said regions.

24. The method of claim 6, wherein the signals produced by the second and third reagents are evaluated to determine the adequacy of the specimen.

25. A method for detecting dysplastic cells in a cervical cytological sample, the method comprising:
collecting the cervical cytology sample;
preparing a cell suspension from the cervical cytology sample;
depositing a representative sampling of the cell suspension on a microscope slide;
fixing the deposited cells on the microscope slide;
contacting the cytological sample with a cytological detection cocktail comprising:
(i) a first reagent that identifies proliferating cells;
(ii) a second reagent that identifies proliferating non-dysplastic cells; and
(iii) a third reagent that identifies cellular material; and
classifying as abnormal any portions of the cytological sample that have been identified by the first reagent and the third reagent but not the second reagent.

26. The method of claim 25, wherein the step of depositing a representative sample of the cell suspension on a microscope slide further comprises a subsequent step of dispersing the cells through chemical and mechanical means.

27. The method of claim 25, wherein the step of depositing a representative sample of the cell suspension on a microscope slide comprises a sedimentation step.

28. The method of claim 25, wherein the step of fixing the deposited cells on the microscope slide comprises applying a fixative by at least one of spraying, smearing or dipping.

29. The method of claim 25, wherein the first reagent comprises an antibody directed to at least one of epidermal growth factor receptor and transferrin receptor, and further comprises a fluorophore.

30. The method of claim 25, wherein the second reagent comprises an antibody specific one or more cytokeratins and further comprises a fluorophore.

31. The method of claim 25, wherein the third reagent comprises a reagent that selectively and differentially stains cellular DNA.

32. The method of claim 31, wherein the third reagent comprises a reagent that binds to cellular DNA in a defined stoichiometric ratio.

33. The method of claim 22, wherein each of the three reagents can be spectrally differentiated from each other on a basis of fluorescent emission, optical absorbance or a combination of fluorescent emission and optical absorbance.

34. A method for detecting abnormal cells in a cervical cytological sample, the method comprising:
collecting the cervical cytology sample using a cell collection device;
fixing the deposited cells on the cell collection device;
contacting the cytological sample with a cytological detection cocktail comprising:
(i) a first reagent that labels proliferating cells;
(ii) a second reagent that labels proliferating non-dysplastic cells; and
(iii) a third reagent that labels cellular material; and
identifying as abnormal any cells that have been labeled by the first reagent and the third reagent but not by the second reagent.

35. The method of claim 34, wherein the first reagent comprises antibodies to epidermal growth factor receptor and transferrin receptor that are each conjugated to fluorophores.

36. The method of claim 34, wherein the second reagent comprises an antibody specific to one or more cytokeratins conjugated to a fluorophore.

37. The method of claim 34, wherein the third reagent comprises a reagent that selectively and differentially stains cellular DNA.

38. The method of claim 34, wherein the third reagent comprises a reagent that stains cellular DNA in a defined stoichiometric ratio.

39. The method of claim 34, wherein each of three reagents can be spectrally differentiated from each other on a basis of fluorescent emission, optical absorbance or a combination of fluorescent emission and optical absorbance.

40. A method for detecting abnormal cells in a cytological specimen, the method comprising:
treating said cytological specimen with a first reagent that specifically labels a cellular constituent that differs in concentration between normal and abnormal cells;
treating said cytological specimen with a second reagent that specifically labels cell types that are not of interest, said cell types being capable of expressing the cellular constituent labeled by the first reagent;
treating said cytological specimen with a third reagent that permits discrimination between cellular and non-cellular material;
passing the treated cytological specimen through a flow cytometer that captures the fluorescent emissions from each individual object in the specimen in at least three channels, one channel corresponding to the emissions from one of the three reagents with which the specimen was treated;
evaluating signals in the three channels in order to estimate the relative levels of staining of the object by the first, second and third reagents; and
classifying the cells labeled with the first reagent and the third reagent, but not with the second reagent as being abnormal.

41. The method of claim 40, wherein a light scattering signal is captured and used as a classification parameter.

42. The method of claim 40, wherein the first reagent and the second reagent each comprise fluorescently labeled antibodies.

43. The method of claim 40, wherein the first reagent comprises a fluorescently labeled antibody directed against epidermal growth factor receptor (EGFR).

44. The method of claim 40, wherein the first reagent comprises a fluorescently labeled antibody directed against Transferrin receptor (TfR).

45. The method of claim 40, wherein the second reagent comprises a fluorescently labeled antibody directed against one or more cytokeratins.

46. The method of claim 40, wherein the third reagent comprises a fluorescent DNA stain.

47. The method of claim 40, wherein the third reagent comprises a quantitative fluorescent DNA stain.

48. The method of claim 47, wherein an intensity of fluorescent emission from the third reagent provides an estimate of the DNA content or ploidy of the cell.

49. The method of claim 48, wherein ploidy is included as a parameter in the classification of a cell as being normal or abnormal.

50. The method of claim 40, wherein the captured fluorescent emissions are corrected for spectral overlap prior to image evaluation

51. The method of claim 40, wherein each of the reagents includes a fluorophore, where the fluorophores are selected such as to minimize the spectral overlap resulting from the fluorophores associated with the second and third reagents into the spectral emissions from the fluorophore associated with the first reagent.

52. The method of claim 40, wherein the data is processed and evaluated in a hue-saturation-luminance (HSL) color space.

53. The method of claim 40, wherein the signals produced by the second and third reagents are evaluated to determine the adequacy of the specimen.

54. A method for selectively identifying dysplastic cervical cells, comprising:
contacting the cytological sample with a cytological detection cocktail comprising:
(i) a first reagent that preferentially stains proliferating cervical cells;
(ii) a second reagent that preferentially stains normal endocervical cells; and
(iii) a third reagent that preferentially stains cellular material;
capturing an image of the cytological sample to form a captured cytological image; and
processing the captured cellular image to identify any dysplastic cells by
(i) excluding from consideration any non-cellular material by excluding portions of the cytological sample that has been not stained by the third reagent;
(ii) selecting for consideration proliferating cells by selecting any remaining portions of the cytological sample that has been stained by the first reagent;
(iii) excluding from consideration normal endocervical cells by excluding any remaining portions of the cytological sample that have been stained by the first reagent and by the second reagent; and
(iv) identifying any remaining portions as containing dysplastic cells.

55. A cytological detection cocktail for selectively identifying dysplastic cells in the presence of non-dysplastic cells, the cytological detection cocktail comprising:
a reagent that selectively and differentially stains proliferating cells;
a reagent that selectively and differentially stains proliferating non-dysplastic cells; and
a reagent that selectively and differentially stains cellular material;
wherein each of the three reagents can be spectrally differentiated from one another.

56. The cytological detection cocktail of claim 55, wherein the reagent that preferntially stains proliferating cells comprises an antibody that is specific to at least one of epidermal growth factor receptor and transferrin receptor and that is conjugated to a fluorophore.

57. The cytological detection cocktail of claim 55, wherein the reagent that preferentially stains proliferating non-dysplastic cells comprises an antibody that is specific to one or more cytokeratins and that is conjugated to a fluorophore.

58. The cytological detection cocktail of claim 55, wherein the reagent that preferentially stains cellular material comprises a reagent that selectively and differentially stains cellular DNA and that is conjugated to a fluorophore.

59. The cytological detection cocktail of claim 55, wherein each of three reagents can be spectrally differentiated from each other on a basis of fluorescent emission, optical absorbance or a combination of fluorescent emission and optical absorbance.

60. A cytological detection kit comprising the cytological detection cocktail of claim 55 in combination with instructions for their use.
